(19) United States
(12) Patent Application Publication
(10) Pub. No.: US 2026/0069610 A1
(43) Pub. Date: Mar. 12, 2026

(54) INHIBITION OF EFFEROCYTOSIS AS A TREATMENT TO PREVENT BONE LOSS AND INCREASE BONE DENSITY AND STRENGTH

(71) Applicant: University of Rochester, Rochester, NY (US)

(72) Inventors: Laura Calvi, Rochester, NY (US); Emily Quarato, Rochester, NY (US); Roman Eliseev, Rochester, NY (US); Michael Becker, Rochester, NY (US)

(73) Assignee: University of Rochester, Rochester, NY (US)

(21) Appl. No.: 19/108,663

(22) PCT Filed: Sep. 5, 2023
(86) PCT No.: PCT/US2023/073461

§ 371 Date: Mar. 4, 2025

Related U.S. Application Data
(60) Provisional application No. 63/404,985, filed on Sep. 9, 2022.

(51) Int. Cl. A61K 31/635; A61K 31/4709; A61K 31/517; A61P 19/10

(52) U.S. Cl. CPC A61K 31/635; A61K 31/4709; A61K 31/517; A61P 19/10

(57) ABSTRACT

This disclosure relates methods and compositions for preventing bone loss and increasing bone density and strength.

### CROSS-REFERENCE TO RELATED APPLICATIONS

This application is a 371 national phase application of International Application No. PCT/US2023/073461, filed Sep. 5, 2023, which claims priority under 35 U.S.C. § 119 (e) to U.S. Provisional Patent Application No. 63/404,985, filed Sep. 9, 2022. The foregoing applications are incorporated by reference herein in their entirety.

### GOVERNMENT INTERESTS

This invention was made with government support under AG076786 awarded by the National Institutes of Health. The government has certain rights in the invention.

### FIELD OF THE INVENTION

This disclosure relates to methods and compositions for preventing bone loss and increasing bone density and strength.

### BACKGROUND OF THE INVENTION

Bone loss can be caused by a wide variety of conditions and may result in significant medical problems. For example, osteoporosis is a debilitating disease in humans and is characterized by marked decreases in skeletal bone mass and mineral density, structural deterioration of bone, including degradation of bone microarchitecture and corresponding increases in bone fragility, decreases in bone strength, and susceptibility to fracture in afflicted individuals. Osteoporosis in humans is generally preceded by clinical osteopenia, a condition found in approximately 25 million people in the United States. Another 7-8 million patients in the United States have been diagnosed with clinical osteoporosis. The frequency of osteoporosis in the human population increases with age. Among Caucasians, osteoporosis is predominant in women who, in the United States, comprise 80% of the osteoporosis patient pool. There is a need for a more effective treatment for preventing bone loss and increasing bone density and strength.

### SUMMARY OF INVENTION

This disclosure addresses the need mentioned above in a number of aspects. In one aspect, this disclosure provides a method of increasing bone density or reducing bone loss or bone resorption in a subject in need thereof. In some embodiments, the method comprises administering to the subject an effective amount of an agent capable of reducing efferocytosis by mesenchymal stromal cells (MSCs).

In another aspect, this disclosure provides a method of treating a disease or disorder associated with reduced bone density resorption in a subject in need thereof. In some embodiments, the method comprises administering to the subject an effective amount of an agent capable of reducing efferocytosis by mesenchymal stromal cells.

In some embodiments, the mesenchymal stromal cells comprise bone marrow mesenchymal stromal cells.

In some embodiments, the agent is a small molecule compound, an oligonucleotide, a nucleic acid, a peptide, a polypeptide, or an antibody or an antigen-binding portion thereof. In some embodiments, the agent comprises an inhibitor of a Tyro3, Axl, and Mer (TAM) receptor kinase or an inhibitor specific to an Axl receptor kinase, or a derivative thereof. In some embodiments, the agent comprises a pan-TAM inhibitor.

In some embodiments, the agent comprises LDC1267, TP0903, bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, or a combination thereof.

In some embodiments, the agent comprises LDC1267 or a derivative thereof, and wherein LDC1267 is represented by the following structure:

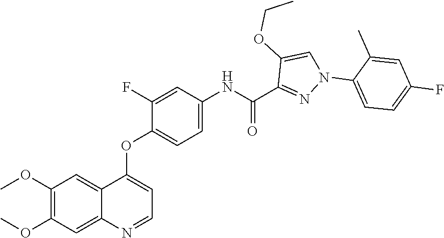 [SMILES: CCOc1cn(-c2ccc(F)cc2C)nc1C(=O)Nc1ccc(Oc2ccnc3cc(OC)c(OC)cc23)c(F)c1]

In some embodiments, the agent comprises TP0903 or a derivative thereof, and wherein TP0903 is represented by the following structure:

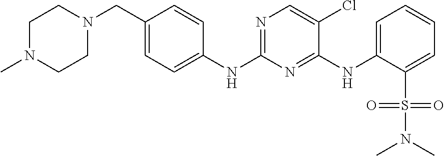 [SMILES: CN1CCN(Cc2ccc(Nc3ncc(Cl)c(Nc4ccccc4S(=O)(=O)N(C)C)n3)cc2)CC1]

In some embodiments, the agent comprises a mitochondrial division inhibitor.

In some embodiments, the agent comprises mitochondrial division inhibitor 1 (Mdivi-1) or a derivative thereof, and wherein Mdivi-1 is represented by the following structure:

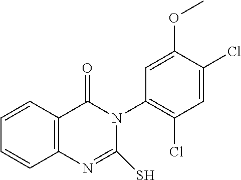 [SMILES: COc1cc(-n2c(S)nc3ccccc3c2=O)c(Cl)cc1Cl]

The method of any one of the preceding claims, wherein treatment by the agent results in 10-15% reduction of bone loss or 10-15% increase of bone density in both young and aged individuals.

In some embodiments, the subject has increased efferocytosis by mesenchymal stromal cells caused by one or more conditions. In some embodiments, the one or more conditions comprise macrophage dysfunction. In some embodiments, the one or more conditions comprise excessive apoptotic cell burden.

In some embodiments, the macrophage dysfunction is caused by aging or a disease selected from cancer, diabetes, obesity, atherosclerosis, and autoimmunity. In some embodiments, the autoimmunity is associated with rheumatoid arthritis, lupus, or osteoarthritis.

In some embodiments, the excessive apoptotic cell burden is caused by radiation, chemotherapy, or an injury.

In some embodiments, the disease or disorder associated with reduced bone density is osteoporosis, a critical sized-bone defect, a mechanical disorder resulting from disuse or injury, osteogenesis imperfecta, osteomalacia, bone necrosis, rickets, osteomyelitis, alveolar bone loss, Paget's disease, hypercalcemia, primary hyperparathyroidism, metastatic bone diseases, myeloma, or bone loss.

In some embodiments, the bone loss is caused by aging, cancer, fibrous dysplasia, aplastic bone diseases, metabolic bone diseases, type 1 diabetes, lupus, rheumatoid arthritis, inflammatory bowel disease, hyperthyroidism, celiac disease, asthma, multiple sclerosis, periodontitis, space travel, or a combination thereof.

In some embodiments, the subject is a mammal. In some embodiments, the subject is a human.

In some embodiments, the agent is administered to the subject at one or more doses of from about 0.1 to about 100 mg/kg of body weight of the subject. In some embodiments, the one or more doses of the agent are administered at least every 1 day, 3 days, 5 days, 1 week, 2 weeks, 3 weeks, or 4 weeks.

In some embodiments, the agent is administered to the subject intratumorally, intravenously, subcutaneously, intraosseously, orally, transdermally, sublingually, in sustained release, in controlled release, in delayed release, or as a suppository.

In some embodiments, the method further comprises administering to the subject an additional therapeutic agent or therapy. In some embodiments, the additional therapeutic agent or therapy comprises a second inhibitor of the TAM receptor kinase or a derivative thereof.

In another aspect, this disclosure also provides use of an agent capable of reducing efferocytosis by mesenchymal stromal cells in the manufacture of a medicament for increasing bone density or reducing bone loss or bone resorption in a subject in need thereof.

In another aspect, the disclosure further provides use of an agent capable of reducing efferocytosis by mesenchymal stromal cells in the manufacture of a medicament for treating a disease or disorder associated with reduced bone density in a subject in need thereof.

In yet another aspect, the disclosure additionally provides a composition for increasing bone density or reducing bone loss or bone resorption in a subject in need thereof or treating a disease or disorder associated with reduced bone density. In some embodiments, the composition comprises an agent capable of reducing efferocytosis by mesenchymal stromal cells.

Also within the scope of this disclosure is a kit for increasing bone density or reducing bone loss or bone resorption in a subject in need thereof or treating a disease or disorder associated with reduced bone density. In some embodiments, the kit comprises an agent capable of reducing efferocytosis and instructional materials.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, and 1C show loss of Axl decreases efferocytic function in mesenchymal stromal cells (MSCs) in vitro. FIGS. 1A and 1B show quantification of percent of efferocytic MSCs and efficiency (MFI) efferocytosis by murine MSCs (mMSCs) incubated with excess (10 PMN: 1 MSC) end stage neutrophils (mPMNs) over 24 hrs. N=3 mice. Mean±SEM shown. Student's t-test, *p<0.05. FIG. 1C shows representative microscopy images showing uptake of end stage mPMNs stained with PKH26 by mMSC.

FIGS. 2A, 2B, and 2C show that genetic loss of Axl decreases efferocytic function and efficiency in BM-MSC in vivo at 3 months of age. Quantification of engulfment of end stage neutrophils by bone marrow MSC by percentage, count, and efficiency (MFI) 18 hr after in vivo injection with 8-10e6 PKH26 stained end stage neutrophils. N=6 mice. Mean±SD shown graph. Student's t-test, ** p<0.01

FIGS. 3A and 3B show that loss of Axl increases bone mineral density and cortical thickness at 3 and 24 months. Quantification and representative μCT images of cortical bone volume over total volume (BV/TV), mineralization density, and cortical thickness of femurs from C57Bl/6 (WT) vs. Axl knockout (Axl KO) mice measured by μCT at 3 and 24 months of age. N=9-12 mice. Mean±SD shown graph. Student's t-test, *p<0.05 or listed p-value

FIGS. 4A and 4B show that pharmacological Axl Inhibitor (TP0903) blocks efferocytosis for a short duration. Quantification of efferocytosis at 3 and 24 hrs by ST2 cells, a bone marrow derived stromal cell line, pre treated with 100 nM Axl inhibitor (TP0903) for 1 hr incubated with excess (5 PMN: 1 MSC) end stage neutrophils (hPMNs). All graphs are shown as Mean±SD, N=2-5, Student's t-test: ** p<0.01.

FIGS. 5A and 5B show that pharmacological Axl+Tyro3+MerTK Inhibitor (LDC1267) blocks efferocytosis in MSCs. Quantification of efferocytosis at 3 and 24 hrs by ST2 cells, a bone marrow-derived stromal cell line, pre-treated with 10 μM Pan-TAM inhibitor (LDC1267) for 1 hr incubated with excess (5 PMN: 1 MSC) end stage neutrophils (hPMNs). All graphs are shown as Mean±SD, N=2-4, Student's t-test: **** p<0.0001.

FIGS. 6A, 6B, 6C, and 6D show that an inhibitor for mitochondrial fission (MDivi) decreases human MSC (hMSC) efferocytosis and restores MSC osteoblastic differentiation. FIGS. 6A, 6B, and 6C show quantification via flow cytometry of viability, end stage neutrophil (PMNs) engulfment, and efficiency (mean fluorescent intensity, MFI) of hMSCs pre-treated with 25 μM Mdivi for 1 h and then given end stage PMN for 24 h. FIG. 6D shows quantification of Alkaline Phosphatase (ALP) via qPCR of hMSCs pre-treated with 25 μM Mdivi for 1 h and then given end stage PMN for 3 h. N=3, Mean±SD shown on graphs ** p<0.01, *** p<0.001, (1-test or ANOVA).

FIGS. 7A and 7B show that MSC efferocytosis is inversely correlated with macrophage number and function. Quantification of efferocytosis and efficiency (MFI) by murine MSCs (mMSCs) incubated in increasing percentage of murine macrophage with excess (10 PMN: 1 MSC) end stage neutrophils (PMNs) over 24 hrs. N=24. Simple linear regression with line of best fit and 95% confidence intervals, p-value 0.0005 and <0.0001, respectively.

FIGS. 8A and 8B show that MSC efferocytosis is increased in aging, where macrophages have known efferocytic defects. FIG. 8A shows quantification of overall end stage neutrophil engulfment by macrophages in young (3-4 m) and aged (20-24 m) mice. N=4-5 mice, Student's t-test, *** p<0.001. Graphs are shown as Mean±SD. FIG. 8B shows quantification of overall end stage neutrophil engulfment by mesenchymal stromal cells (MSC, lineage⁻CD45⁻CD31⁻CD51⁺Sca1⁺) populations in young (3-4 m) and aged (20-24 m) mice. N=4-10 mice, ANOVA, *p<0.05. Graphs are shown as Mean±SEM.

FIGS. 9A and 9B show that radiation increases MSC efferocytic potential and efficiency in vitro. Mice were given total body irradiation at 6.5Gy 2×, for a total of 13Gy. MSC were harvested for in vitro analysis. Quantification of efferocytosis and efficiency (MFI) by murine MSCs (mMSCs) incubated with excess (10 PMN: 1 MSC) end stage neutrophils (mPMNs) over 24 hrs. N=3 mice, Student's t-test, *p<0.05, ** p<0.01. Graphs are shown as Mean±SD.

FIGS. 10A and 10B show that radiation increases MSC number and cellular engulfment. Mice were given total body irradiation at 6.5Gy 1× and then dosed with 8-10×10⁶ PKH26 stained end stained neutrophils for 18 hours. Quantification of the total number of MSCs and engulfment of end stage neutrophils by bone marrow MSC after the 18 hr incubation. N=3-5 mice, Student's t-test, *p<0.05, ** p<0.01. Graphs are shown as Mean±SD.

### DETAILED DESCRIPTION OF THE INVENTION

This disclosure relates to an unexpected discovery that agents capable of reducing efferocytosis by mesenchymal stromal cells (MSCs), such as Tyro3, Axl, and Mer (TAM) receptor kinase inhibitors, are effective in treating or alleviating conditions associated with efferocytosis by MSCs. In patients who have been diagnosed with the condition, the methods of this disclosure can, e.g., reverse or inhibit the worsening of symptoms related to such condition and/or prevent development of new symptoms.

### Methods of Treating Bone-Related Conditions

Accordingly, in one aspect, this disclosure provides a method of increasing bone density or reducing bone loss or bone resorption in a subject in need thereof. In some embodiments, the method comprises administering to the subject an effective amount of an agent capable of reducing efferocytosis by MSCs (e.g., bone marrow MSCs).

In another aspect, this disclosure provides a method of treating a disease or disorder associated with reduced bone density resorption in a subject in need thereof. In some embodiments, the method comprises administering to the subject an effective amount of an agent capable of reducing efferocytosis by MSCs (e.g., bone marrow MSCs).

As used herein, the term “mesenchymal stromal cells,” “mesenchymal stem cells,” or “MSCs” refers to multipotent stem cells, which can differentiate into a variety of cell types, including, for example, osteoblasts, chondrocytes, and adipocytes, etc. The mesenchymal stem cells or MSCs may be derived from any tissue sources, including but not limited to bone marrow tissues, adipose tissue, muscle tissue, corneal stroma or dental pulp of deciduous baby teeth, umbilical cord tissues or umbilical cord blood, etc. In some embodiments, the MSCs can be bone marrow MSCs.

As used herein, “efferocytosis” refers to effective clearance of apoptotic cells by professional and non-professional phagocytes. The process is mechanically different from other forms of phagocytosis and involves the localization, binding, internalization, and degradation of apoptotic cells.

As used herein, a “disease associated with reduced bone density” refers to a condition characterized by increased bone porosity resulting from either (1) abnormally decreased bone deposition (e.g., formation) relative to a healthy individual (e.g., a subject not having a disease characterized by decreased bone density), or (2) abnormally increased bone resorption (e.g., breakdown) relative to a healthy individual (e.g., a subject not having a disease characterized by decreased bone density). A disease associated with increased bone porosity may arise from either (1) abnormally decreased osteoblast and/or osteocyte differentiation, function, or activity relative to a healthy individual (e.g., a subject not having a disease characterized by decreased bone density) and/or (2) abnormally increased osteoclast differentiation, function, or activity relative to a healthy individual (e.g., a subject not having a disease characterized by decreased bone density). “Porosity” generally refers to the volume of fraction of bone not occupied by bone tissue.

In some embodiments, examples of bone-related conditions include achondroplasia, cleidocranial dysostosis, enchondromatosis, fibrous dysplasia, Gaucher's Disease, hypophosphatemic rickets, Marfan's syndrome, multiple hereditary exotoses, neurofibromatosis, osteogenesis imperfecta, osteopetrosis, osteopoikilosis, sclerotic lesions, pseudoarthrosis, pyogenic osteomyelitis, periodontal disease, anti-epileptic drug induced bone loss, primary and secondary hyperparathyroidism, familial hyperparathyroidism syndromes, weightlessness induced bone loss, osteoporosis in men, postmenopausal bone loss, osteoarthritis, renal osteodystrophy, infiltrative disorders of bone, oral bone loss, osteonecrosis of the jaw, juvenile Paget's disease, melorheostosis, metabolic bone diseases, mastocytosis, sickle cell anemia/disease, organ transplant related bone loss, kidney transplant related bone loss, systemic lupus erythematosus, ankylosing spondylitis, epilepsy, juvenile arthritides, thalassemia, mucopolysaccharidoses, Fabry Disease, Turner Syndrome, Down Syndrome, Klinefelter Syndrome, leprosy, Perthes' Disease, adolescent idiopathic scoliosis, infantile onset multi-system inflammatory disease, Winchester Syndrome, Menkes Disease, Wilson's Disease, ischemic bone disease, Legg-Calve-Perthes disease, regional migratory osteoporosis, anemic states, conditions caused by steroids, glucocorticoid-induced bone loss, heparin-induced bone loss, bone marrow disorders, scurvy, malnutrition, calcium deficiency, osteoporosis, osteopenia, alcoholism, chronic liver disease, postmenopausal state, chronic inflammatory conditions, rheumatoid arthritis, inflammatory bowel disease, ulcerative colitis, inflammatory colitis, Crohn's disease, oligomenorrhea, amenorrhea, pregnancy, diabetes mellitus, hyperthyroidism, thyroid disorders, parathyroid disorders, Cushing's disease, acromegaly, hypogonadism, immobilization or disuse, reflex sympathetic dystrophy syndrome, regional osteoporosis, osteomalacia, bone loss associated with joint replacement, HIV associated bone loss, bone loss associated with loss of growth hormone, bone loss associated with cystic fibrosis, chemotherapy associated bone loss, tumor induced bone loss, cancer-related bone loss, hormone ablative bone loss, multiple myeloma, drug-induced bone loss, anorexia nervosa, disease associated facial bone loss, disease associated cranial bone loss, disease associated boneloss of the jaw, disease associated bone loss of the skull, bone loss associated with aging, facial bone loss associated with aging, cranial bone loss associated with aging, jaw bone loss associated with aging, skull bone loss associated with aging, and bone loss associated with space travel.

In some embodiments, the subject has increased efferocytosis by mesenchymal stromal cells caused by one or more conditions. In some embodiments, one or more conditions comprise macrophage dysfunction (e.g., defective macrophage). In some embodiments, one or more conditions comprise excessive apoptotic cell burden.

In some embodiments, macrophage dysfunction is caused by aging or a disease selected from cancer, diabetes, obesity, atherosclerosis, and autoimmunity (e.g., autoimmune disease). In some embodiments, the autoimmunity is associated with rheumatoid arthritis, lupus, or osteoarthritis. In some embodiments, the autoimmune disease is immune neutropenia, Guillain-Barré syndrome, epilepsy, autoimmune encephalitis, Isaac's syndrome, nevus syndrome, Pemphigus vulgaris, Pemphigus foliaceus, Bullous pemphigoid, epidermolysis bullosa acquisita, pemphigoid gestationis, mucous membrane pemphigoid, antiphospholipid syndrome, autoimmune anemia, autoimmune Grave's disease, Goodpasture's syndrome, myasthenia gravis, multiple sclerosis, rheumatoid arthritis, lupus, idiopathic thrombocytopenic purpura, lupus nephritis, or membranous nephropathy.

In some embodiments, the excessive apoptotic cell burden is caused by radiation, chemotherapy, or an injury (e.g., bone fracture).

In some embodiments, the disease or disorder associated with reduced bone density is osteoporosis, a critical sized-bone defect, a mechanical disorder resulting from disuse or injury, osteogenesis imperfecta, osteomalacia, bone necrosis, rickets, osteomyelitis, alveolar bone loss, Paget's disease, hypercalcemia, primary hyperparathyroidism, metastatic bone diseases, myeloma, or bone loss.

In some embodiments, the bone loss is caused by aging, cancer, fibrous dysplasia, aplastic bone diseases, metabolic bone diseases, type 1 diabetes, lupus, rheumatoid arthritis, inflammatory bowel disease, hyperthyroidism, celiac disease, asthma, multiple sclerosis, periodontitis, space travel, or a combination thereof.

An “inhibitor” or “antagonist” of a polypeptide or a signal transduction pathway is an agent that reduces, by any mechanism, any polypeptide action, as compared to that observed in the absence (or presence of a smaller amount) of the agent. An inhibitor of a polypeptide or a signal transduction pathway can affect: (1) the expression, mRNA stability, protein trafficking, modification (e.g., phosphorylation), or degradation of the polypeptide or a component of the signal transduction pathway, or (2) one or more of the normal functions of the polypeptide or a component of the signal transduction pathway. An inhibitor of a polypeptide or a component of the signal transduction pathway can be non-selective or selective. In some embodiments, inhibitors/antagonists can include small or large molecules that act directly on, and are selective for, the target polypeptide.

The terms “inhibit” and “antagonize,” as used herein, mean to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or downregulate a protein, a gene, and an mRNA stability, expression, function, and activity, e.g., antagonists.

As used herein, the terms “increase,” “elevate,” “elevated,” “upregulate,” “enhance,” and “activate” all generally refer to an increase by a statically significant amount as compared to a reference level (e.g., a reference expression level). For the avoidance of any doubt, these terms mean an increase of at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, an increase of at least about 10%, at least about 20%, at least about 30%, at least about 40%, at least about 50%, at least about 60%, at least about 70%, at least about 80%, at least about 90% or at least about 100%, as compared to a reference level.

As used herein, the terms “decrease,” “reduce,” “downregulate,” and “inhibit” all generally refer to a decrease by a statistically significant amount. However, for avoidance of doubt, the term “reduced,” “decrease,” “reduce,” or “inhibit” means a decrease by at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, a decrease by at least about 10%, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease of 10-100% as compared to a reference level.

In some embodiments, the agent is a small molecule compound, an oligonucleotide, a nucleic acid, a peptide, a polypeptide, or an antibody or an antigen-binding portion thereof.

In some embodiments, the agent inhibits one or more of Axl, Tyro3, MerTK, Igtav, Megf10, and related pathways.

In some embodiments, the agent comprises an inhibitor of a Tyro3, Axl, and Mer (TAM) receptor kinase or an inhibitor specific to an Axl receptor kinase, or a derivative thereof. In some embodiments, the agent comprises a pan-TAM inhibitor.

In some embodiments, the agent comprises LDC1267, TP0903, bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, MP470, SGI-AXL-277, AXL-1, AXL-2, AXL-3, AXL-4, AXL-5, AXL-6, AXL-7, AXL-8, AXL-9, Mdivi-1, or a combination thereof, e.g., as described in PCT Publication No: WO2009062112A2, WO2019113190A1, WO2017035366A1, or WO2017172596A1, the disclosures of which are incorporated herein by reference. Other specific examples of TAM receptor inhibitors can be found in PCT Publication Nos: WO07030680A3, WO06052936A3, WO04092735A3, WO07056151A2, and U.S. Patent Publication No: US20070142402, the disclosures of which are incorporated herein by reference.

In some embodiments, an inhibitor of a TAM receptor kinase has an IC50 of less than about 50 μM against Tyro3, Axl, and/or Mer receptor.

In some embodiments, the agent comprises LDC1267 or a derivative thereof. LDC1267 is a highly selective TAM kinase inhibitor with IC₅₀s of <5 nM/8 nM/29 nM for Tyro3, Axl, and Mer, respectively. LDC1267 is represented by the following structure:

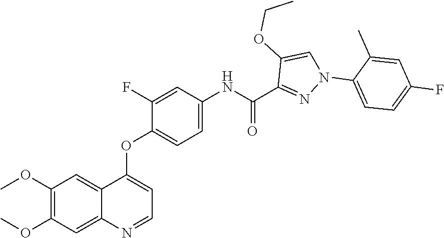 [SMILES: CCOc1cn(-c2ccc(F)cc2C)nc1C(=O)Nc1ccc(Oc2ccnc3cc(OC)c(OC)cc23)c(F)c1]

In some embodiments, the agent comprises Dubermatinib (TP-0903) or a derivative thereof. Dubermatinib is a potent and selective Axl receptor tyrosine kinase inhibitor with an IC₅₀ value of 27 nM. Dubermatinib is represented by the following structure:

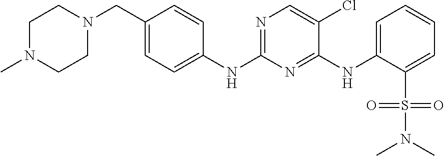 [SMILES: CN1CCN(Cc2ccc(Nc3ncc(Cl)c(Nc4ccccc4S(=O)(=O)N(C)C)n3)cc2)CC1]

In some embodiments, the agent comprises a mitochondrial division inhibitor. In some embodiments, the agent comprises mitochondrial division inhibitor 1 (Mdivi-1) or a derivative thereof. Mdivi-1 is a selective dynamin-related protein 1 (Drp1) inhibitor and a mitochondrial division/mitophagy inhibitor. Mdivi-1 is represented by the following structure:

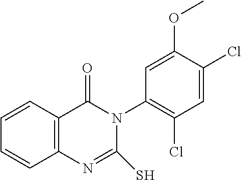 [SMILES: COc1cc(-n2c(S)nc3ccccc3c2=O)c(Cl)cc1Cl]

As used herein, a “derivative” of an agent includes, without limitation, a stereoisomer, an analog, a prodrug, a metabolite, or a pharmaceutically acceptable salt of the agent, or a combination thereof, that is suitable for use in the disclosed methods.

In some embodiments, a “derivative” refers to a chemical substance related structurally to another, i.e., an “original” substance, which can be referred to as a “parent” compound. A “derivative” can be made from the structurally related parent compound in one or more steps. The phrase “closely related derivative” means a derivative whose molecular weight does not exceed the weight of the parent compound by more than 50%. The general physical and chemical properties of a closely related derivative are also similar to the parent compound. “Pharmaceutically active derivative” refers to any compound that, upon administration to the recipient, is capable of providing, directly or indirectly, the activity disclosed herein.

As used herein, “isomers” are different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either (R) or(S). Resolved compounds whose absolute configuration is unknown can be designated (+) or (−) depending on the direction (dextro- or levorotatory) in which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or(S). The present chemical entities, pharmaceutical compositions, and methods are meant to include all such possible isomers, including racemic mixtures, optically pure forms, and intermediate mixtures. Optically active (R)- and (S)-isomers can be prepared using chiral synthons or chiral reagents or resolved using conventional techniques. When the compounds described herein contain olefinic double bonds or other centers of geometric asymmetry, and unless specified otherwise, it is intended that the compounds include both E and Z geometric isomers.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related to mirror images, most commonly because they contain two or more asymmetrically substituted carbon atoms. “R” and “S” represent the configuration of substituents around one or more chiral carbon atoms. Thus, “R*” and “S*” denote the relative configurations of substituents around one or more chiral carbon atoms. The symbol “*” in a structural formula represents the presence of a chiral carbon center.

As used herein, “racemate” or “racemic mixture” means a compound of equimolar quantities of two enantiomers, wherein such mixtures exhibit no optical activity, i.e., they do not rotate the plane of polarized light.

As used herein, “geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “St,” “R*,” “E,” “Z,” “cis,” and “trans” indicate configurations relative to the core molecule.

An “analog” refers to a small organic compound, a nucleotide, a protein, or a polypeptide that possesses similar or identical activity or function(s) as the compound, nucleotide, protein, or polypeptide or compound having the desired activity of this disclosure, but need not necessarily include a sequence or structure that is similar or identical to the sequence or structure of the preferred embodiments.

A “prodrug” refers to a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers the advantages of solubility, tissue compatibility, or delayed release in a mammalian organism (see, e.g., Bundgaard, H., Design of Prodrugs (1985) (Elsevier, Amsterdam). The term “prodrug” also refers to any covalently bonded carriers, which release the active compound in vivo when administered to a subject. Prodrugs of an active compound, as described herein, may be prepared by modifying functional groups present in the active compound in such a way that the modifications are cleaved, either in routine manipulation or in vivo, to yield the active parent compound. Prodrugs include, for example, compounds wherein a hydroxy, amino or mercapto group is bonded to any group that, when the prodrug of the active compound is administered to a mammalian subject, cleaves to form a free hydroxy, free amino, or free mercapto group, respectively. Examples of prodrugs include, but are not limited to, acetates, ormats, and benzoate derivatives of alcohol, various ester derivatives of a carboxylic acid, or acetamide, formamide, and benzamide derivatives of an amine functional group in the active compound. Various forms of prodrugs are well known in the art and are described in: (a) The Practice of Medicinal Chemistry, Camille G. Wermuth et al., Ch 31, (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds. Ch 5, pages 113-191 (Harwood Academic Publishers, 1991); and (d) Hydrolysis in Drug and Prodrug Metabolism, Bernard Testa and Joachim M. Mayer, (Wiley-VCH, 2003).

As used herein, the term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable salt” refers to a salt of the administered compounds prepared from pharmaceutically acceptable non-toxic acids, including inorganic acids, organic acids, solvates, hydrates, or clathrates thereof.

The above-described agents can be administered by any suitable means known in the art. When used for human and veterinary treatment, the amount of a particular agent that is administered may be dependent on a variety of factors. Examples of these factors include the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein may be administered in a therapeutically effective amount to a subject in need of treatment. Administration of compositions described herein can be via any suitable route of administration, which may include ingestion, or alternatively parenterally, for example, intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflation, or topical application Such administration may be as single or multiple oral doses, a defined number of eardrops, or a bolus injection, multiple injections, or as a short- or long-duration infusion. Implantable devices (e.g., implantable infusion pumps) may also be employed for the periodic parenteral delivery over time of equivalent or varying dosages of the particular composition. For such parenteral administration, the compositions are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives. The preparation of suitable and sterile parenteral compositions is described in detail in the section entitled “Compositions” above. Compositions described herein can be administered by a number of methods sufficient to deliver the composition to a target tissue or site.

In some embodiments, the agent is administered to the subject intratumorally, intravenously, subcutaneously, intraosseously, orally, transdermally, sublingually, in sustained release, in controlled release, in delayed release, or as a suppository.

As used herein, an “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. A “therapeutically effective amount” of a compound with respect to the subject method of treatment refers to an amount of the compound in a preparation which, when administered as part of a desired dosage regimen (to a mammal, e.g., a human) alleviates a symptom, ameliorates a condition, or slows the onset of disease conditions according to clinically acceptable standards for the disorder or condition to be treated, e.g., at a reasonable benefit/risk ratio applicable to any medical treatment.

The agent can be conveniently administered in a unit dosage form, for example, containing 5 to 1000 mg/m², conveniently 10 to 750 mg/m², or 50 to 500 mg/m² of active ingredient per unit dosage form. In some embodiments, a dose may be presented in a single dose or as divided doses administered at appropriate intervals, for example, as two, three, four, or more sub-doses per day. The sub-dose itself may be further divided, e.g., into a number of discrete, loosely spaced administrations.

The actual dosage amount of an agent or a composition thereof administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In some embodiments, the method comprises administering to the subject one or more doses of the agent comprising from 0.1 mg/kg/body weight to about 1000 mg/kg/body weight or more, e.g., about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more, or any range derivable therein.

In some embodiments, the agent is administered to the subject at one or more doses of from about 0.01 to about 100 mg/kg (e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, or about 99 mg/kg) of body weight of the subject. In some embodiments, the agent is administered at one or more doses of from about 1 to about 50 mg/kg of body weight of the subject. In some embodiments, the agent is administered at one or more doses of from about 8 to about 16 mg/kg of body weight of the subject.

In some embodiments, the agent is administered to the subject at one or more doses of from about 0.1 to about 100 mg/kg (e.g., 0.1, 0.5, 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 100 mg/kg) of body weight of the subject.

In some embodiments, one or more doses of the agent are administered at least every 1 day, every 2 days, every 3 days, every 4 days, every 5 days, every 6 days, every 7 days, every 8 days, every 9 days, every 10 days, every 11 days, every 12 days, every 13 days, every 14 days, every 15 days, every 16 days, every 17 days, every 18 days, every 19 days, every 20 days, every 21 days, every 22 days, every 23 days, every 24 days, every 25 days, every 26 days, every 27 days, every 28 days, every 29 days, every 30 days, every 31 days, every 32 days, every 33 days, every 34 days, every 35 days, every 36 days, every 37 days, every 38 days, every 39 days, every 40 days, every 41 days, every 42 days, every 43 days, every 44 days, every 45 days, every 46 days, every 47 days, every 48 days, every 49 days, every 50 days, every 51 days, every 52 days, every 53 days, every 54 days, every 55 days, every 56 days, every 57 days, every 58 days, every 59 days, every 60 days, every 61 days, every 62 days, every 63 days, every 64 days, every 65 days, every 66 days, every 67 days, every 68 days, every 69 days, every 70 days, every 71 days, every 72 days, every 73 days, every 74 days, every 75 days, every 76 days, every 77 days, every 78 days, every 79 days, every 80 days, every 81 days, every 82 days, every 83 days, every 84 days, every 85 days, every 86 days, every 87 days, every 88 days, every 89 days, every 90 days, every 91 days, every 92 days, every 93 days, every 94 days, every 95 days, every 96 days, every 97 days, every 98 days, every 99 days, every 100 days, every 101 days, every 102 days, every 103 days, every 104 days, every 105 days, every 106 days, every 107 days, every 108 days, every 109 days, every 110 days, every 111 days, every 112 days, every 113 days, every 114 days, every 115 days, every 116 days, every 117 days, every 118 days, every 119 days, or every 120 days.

In some embodiments, one or more doses of the agent are administered at least every 1 day, 3 days, 5 days, 1 week, 2 weeks, 3 weeks, or 4 weeks.

In some embodiments, the treatment produces a therapeutic effect selected from reduced bone loss, reduced bone reabsorption, and increased bone density, as well as improvement in other bone-related conditions (e.g., disorders or diseases). The method of any one of the preceding claims, wherein treatment by the agent results in 10-15% reduction of bone loss or 10-15% increase of bone density.

As used herein, the term “therapeutic effect” is art-recognized and refers to a local or systemic effect in animals, particularly mammals, and more particularly humans caused by a pharmacologically active substance.

The agents described herein (e.g., inhibitors of TAM kinase receptors) can also be used in combination with other active ingredients, e.g., another TAM kinase receptor inhibitor. Such combinations can be selected based on the condition to be treated, cross-reactivities of ingredients, and pharmaco-properties of the combination.

Examples of other active ingredients may include LDC1267, TP0903, bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, MP470, SGI-AXL-277, AXL-1, AXL-2, AXL-3, AXL-4, AXL-5, AXL-6, AXL-7, AXL-8, AXL-9, Mdivi-1, or a combination thereof.

As used herein, “combination” therapy, unless otherwise clear from the context, refers to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

It is also possible to combine a compound of the invention with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide synergy and be synergistic, i.e., the effect achieved when the active ingredients used together are greater than the sum of the effects that result from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills, or capsules, or by different injections in separate syringes. In general, during alternation therapy, an effective dosage of each active ingredient is administered sequentially, i.e., serially, whereas, in combination therapy, effective dosages of two or more active ingredients are administered together. A synergistic effect denotes an effect that is greater than the predicted purely additive effects of the individual compounds of the combination.

Combination therapy is further described by U.S. Pat. Nos. 11,103,514, 10,702,495, 9,382,215, and 6,833,373, which include additional active agents that can be combined with the compounds described herein, and additional types of ailments and other conditions that can be treated with a compound or combination of compounds described herein.

In some embodiments, the agent may precede or follow treatment of the other agent by intervals ranging from minutes to weeks. In embodiments where the other agent and expression construct are applied separately to a cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with the disclosed active.

In some embodiments, one or more agents may be administered within about 1 minute, about 5 minutes, about 10 minutes, about 20 minutes, about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the disclosed active agent. In certain other embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the disclosed active. In some situations, it may be desirable to extend the time period for treatment significantly; however, where several weeks (e.g., about 1, about 2, about 3, about 4, about 6, or about 8 weeks or more) lapse between the respective administrations.

Administration of the compositions of the invention to a patient will follow general protocols for the administration of therapeutics, taking into account the toxicity, if any. It is expected that the treatment cycles can be repeated as necessary. It also is contemplated that various standard therapies or adjunct therapies, as well as surgical intervention, may be applied in combination with the described active agent. These therapies include but are not limited to chemotherapy, radiotherapy, immunotherapy, gene therapy, and surgery.

### Compositions and Kits

Also within the scope of this disclosure is a composition or kit for increasing bone density or reducing bone loss or bone resorption in a subject in need thereof or treating a disease or disorder associated with reduced bone density. In some embodiments, the composition comprises an agent capable of reducing efferocytosis and instructional materials. In some embodiments, the kit comprises an agent capable of reducing efferocytosis and instructional materials.

In some embodiments, the agent inhibits one or more of Axl, Tyro3, MerTK, Igtav, Megf10, and related pathways. In some embodiments, the agent comprises an inhibitor of a Tyro3, Axl, and Mer (TAM) receptor kinase or an inhibitor specific to an Axl receptor kinase, or a derivative thereof. In some embodiments, the agent comprises a pan-TAM inhibitor.

In some embodiments, the agent comprises LDC1267, TP0903, bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, MP470, SGI-AXL-277, AXL-1, AXL-2, AXL-3, AXL-4, AXL-5, AXL-6, AXL-7, AXL-8, AXL-9, Mdivi-1, or a combination thereof.

In some embodiments, the composition can further comprise additional agents that can regulate bone marrow mesenchymal stromal cell efferocytosis, inhibit bone resorption, or increase bone density, or prevent bone loss. The compositions described herein can be formulated in any manner suitable for a desired delivery route. Typically, formulations include all physiologically acceptable compositions, including derivatives or prodrugs, solvates, stereoisomers, racemates, or tautomers thereof, with any physiologically acceptable carriers, diluents, and/or excipients.

In some embodiments, the inhibitor is an interfering nucleic acid, such as siRNA, shRNA, miRNA, antisense oligonucleotides (ASOs), and/or a nucleic acid comprising one or more modified nucleic acid residues. In some embodiments, the interfering nucleic acid is optimized (based on sequence) or chemically modified to minimize degradation prior to and/or upon delivery to the tissue of interest. In some embodiments, such optimizations and/or modifications may be made to ensure a sufficient payload of the interfering nucleic acid is delivered to the tissue of interest. Other embodiments include the use of small molecules, aptamers, or oligonucleotides designed to decrease the expression of a gene in the above-mentioned pathway by either binding to a gene's DNA to limit expression, e.g., antisense oligonucleotides, or impose post-transcriptional gene silencing (PTGS) through mechanisms that include, but are not limited to, binding directly to the targeted transcript or gene product or one or more other proteins in such a way that said gene's expression is reduced; or the use of other small molecule decoys that reduce the specific gene's expression.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), and other oligomeric compounds or oligonucleotide mimetics which hybridize to at least a portion of the target nucleic acid and modulate its function. In some embodiments, the inhibitory nucleic acids include antisense oligonucleotides, e.g., antisense RNA, antisense DNA, chimeric antisense oligonucleotides, or antisense oligonucleotides comprising modified linkages or nucleotide; interfering RNA (RNAi), e.g., small interfering RNA (siRNA), or a short hairpin RNA (shRNA); or combinations thereof. The inhibitory nucleic acids can be modified, e.g., to include a modified nucleotide (e.g., locked nucleic acid) or backbone (e.g., backbones that do not include a phosphorus atom therein), or can by mixmers or gapmers; see, e.g., WO2013/006619, which is incorporated herein by reference for its teachings related to modifications of oligonucleotides. Suitable siRNAs directed against a target can be obtained commercially from vendors such as Origene and Santa Cruz Biotechnology, Inc.

In some embodiments, in addition to the composition, the kit may include an additional therapeutic agent. In some embodiments, the additional therapeutic agent is selected from bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, MP470, SGI-AXL-277, AXL-1, AXL-2, AXL-3, AXL-4, AXL-5, AXL-6, AXL-7, AXL-8, AXL-9, Mdivi-1, or a combination thereof.

In some embodiments, the kit also includes a container that contains the composition and optionally informational material. The informational material can be descriptive, instructional, marketing, or other material that relates to the methods described herein and/or the use of the agents for therapeutic benefit. In an embodiment, the kit also includes an additional therapeutic agent, as described herein. For example, the kit includes a first container that contains the composition and a second container for the additional therapeutic agent.

The informational material of the kits is not limited in its form. In some embodiments, the informational material can include information about production of the composition, concentration, date of expiration, batch or production site information, and so forth. In some embodiments, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, audio recording, or information that contains a link or address to substantive material.

The kit can include one or more containers for the composition. In some embodiments, the kit contains separate containers, dividers, or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading.

### Additional Definitions

As used herein, the term “agent” refers to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder, or pathological condition. The term “formulation,” in general, refers to a preparation that includes at least one pharmaceutically active compound optionally in combination with one or more excipients or other pharmaceutical additives for administration to a subject. In general, particular excipients and/or other pharmaceutical additives are typically selected with the aim of enabling a desired stability, release, distribution, and activity of active compound(s) for applications.

As used herein, the term “small molecule” refers to an organic molecule having a molecular weight between 50 Daltons to 2500 Daltons.

As used herein, the term “effective amount” or “therapeutically effective amount” of a compound refers to an amount of the compound that will elicit the biological or medical response of a subject, or ameliorate symptoms, slow or delay disease progression, or prevent a disease, etc. In one embodiment, the term refers to the amount that inhibits or reduces bone absorption or bone loss or that increases bone density. When applied to an individual active ingredient, administered alone, the term refers to that ingredient alone. When applied to a combination, the term refers to combined amounts of the active ingredients that result in the therapeutic effect, whether administered in combination, serially or simultaneously. In one embodiment, the term refers to the individual dosage amounts or ranges of dosage amounts described in the present application.

As used herein, an “inhibitory nucleic acid” is a double-stranded RNA, RNA interference, miRNA, siRNA, shRNA, or antisense RNA, or a portion thereof, or a mimetic thereof, that when administered to a mammalian cell, results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. Typically, expression of a target gene is reduced by 10%, 25%, 50%, 75%, or even 90-100%.

As used herein, the term “siRNA” intends a double-stranded RNA molecule that interferes with the expression of a specific gene or genes post-transcription. In some embodiments, the siRNA functions to interfere with or inhibit gene expression using the RNA interference pathway. Similar interfering or inhibiting effects may be achieved with one or more of short hairpin RNA (shRNA), microRNA (mRNA) and/or nucleic acids (such as siRNA, shRNA, or miRNA) comprising one or more modified nucleic acid residue, e.g. peptide nucleic acids (PNA), locked nucleic acids (LNA), unlocked nucleic acids (UNA), or triazole-linked DNA. Optimally, a siRNA is 18, 19, 20, 21, 22, 23 or 24 nucleotides in length and has a 2-base overhang at its 3′ end. These dsRNAs can be introduced to an individual cell or culture system. Such siRNAs are used to downregulate mRNA levels or promoter activity.

A “subject” refers to a human and a non-human animal. Examples of a non-human animal include all vertebrates, e.g., mammals, such as non-human mammals, non-human primates (particularly higher primates), dog, rodent (e.g., mouse or rat), guinea pig, cat, and rabbit, and non-mammals, such as birds, amphibians, reptiles, etc. In one embodiment, the subject is a human. In another embodiment, the subject is an experimental, non-human animal or animal suitable as a disease model.

The term “therapeutic agent” refers to any agent that is used to treat a disease. A therapeutic agent may be, for example, a polypeptide(s) (e.g., an antibody, an immunoadhesin or a peptibody), an aptamer or a small molecule that can bind to a protein or a nucleic acid molecule that can bind to a nucleic acid molecule encoding a target (i.e., siRNA), etc.

The term “pharmaceutical composition” refers to the combination of an active agent with a carrier, inert or active, making the composition especially suitable for diagnostic or therapeutic use in vivo or ex vivo.

As used herein, the term “pharmaceutically acceptable” refers to a material, such as a carrier or diluent, which does not abrogate the biological activity or properties of the composition and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “cancer” refers to a disease characterized by the uncontrolled (and often rapid) growth of aberrant cells. Cancer cells can spread locally or through the bloodstream and lymphatic system to other parts of the body. Examples of various cancers are described herein and include, but are not limited to, breast cancer, prostate cancer, ovarian cancer, cervical cancer, skin cancer, pancreatic cancer, colorectal cancer, renal cancer, liver cancer, brain cancer, adrenal gland cancer, autonomic ganglial cancer, biliary tract cancer, bone cancer, endometrial cancer, eye cancer, fallopian tube cancer, genital tract cancers, large intestinal cancer, cancer of the meninges, oesophageal cancer, peritoneal cancer, pituitary cancer, penile cancer, placental cancer, pleura cancer, salivary gland cancer, small intestinal cancer, stomach cancer, testicular cancer, thymus cancer, thyroid cancer, upper aerodigestive cancers, urinary tract cancer, vaginal cancer, vulva cancer, lymphoma, leukemia, lung cancer and the like.

As used herein, the term “tumor” is used interchangeably with the term “cancer” herein, e.g., both terms encompass solid and liquid, e.g., diffuse or circulating, tumors. As used herein, the term “cancer” or “tumor” includes premalignant, as well as malignant cancers and tumors.

As used herein, the term “treating” or “treatment” of any disease or disorder refers, In some embodiments, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically (e.g., stabilization of a discernible symptom), physiologically (e.g., stabilization of a physical parameter), or both. In yet another embodiment, “treating” or “treatment” refers to preventing or delaying the onset or development or progression of the disease or disorder. As used herein, “treating” or “treatment” also refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

The terms “prevent,” “preventing,” “prevention,” “prophylactic treatment,” and the like refer to reducing the probability of developing a disorder or condition in a subject, who does not have, but is at risk of or susceptible to developing a disorder or condition.

The term “ameliorate,” as used herein, refers to the effects of administering an agent to a patient (e.g., a myotonic dystrophy patent) that result in any indicia of success in the prevention, reduction, or reversal of one or more symptoms related to the condition. Reduction may be indicated in lesser severity, delayed onset of symptoms, or a slowing of disease progression. The prevention, reduction, or reversal of symptoms can be measured based on objective parameters, such as the results of a physical examination or laboratory test (i.e., blood test), decreased need for medication, decreased need for supportive measures (i.e., use of a ventilator), or increase in mobility. The prevention, reduction, or reversal of symptoms can also be measured based on subjective parameters, such as a reduction in pain or stiffness or an increase in a patient's mobility and sense of wellbeing.

Doses are often expressed in relation to body weight. Thus, a dose which is expressed as [g, mg, or other unit]/kg (or g, mg, etc.) usually refers to [g, mg, or other unit] “per kg (or g, mg, etc.) bodyweight,” even if the term “bodyweight” is not explicitly mentioned.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition that is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order unless noted otherwise.

In cases in which a method may include a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Loss of Axl Decreases Bone Marrow Mesenchymal Stromal Cell Efferocytosis and Increases Bone Density in Aged Mice.

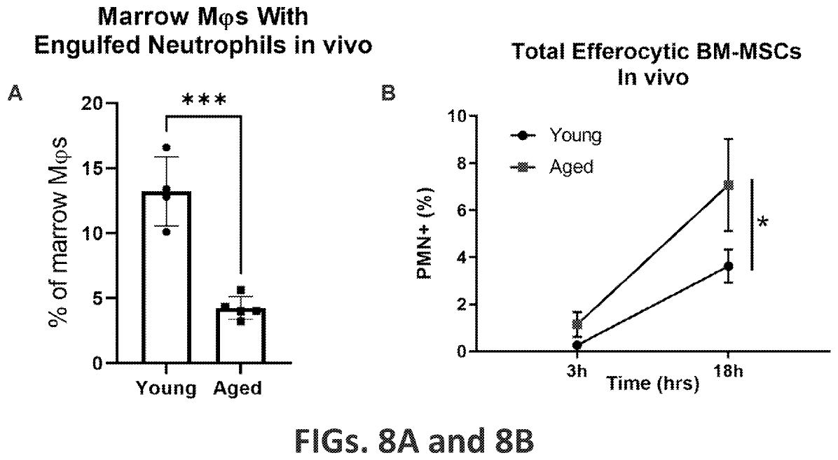

Aging induces osteopenia and increases the risk for fractures and the associated morbidity, and mortality. The aging mechanisms of bone marrow microenvironment components, particularly mesenchymal stromal cells (MSCs) and how they lead to bone loss during aging remain incompletely understood. Studies have shown that MSC senescence contributes to age-dependent bone loss. However, the mechanisms inducing senescence in MSCs during aging remain unclear. It was found that bone marrow-derived macrophages become deficient in their ability to phagocytose dead cells (efferocytosis) during aging (FIG. 8A). Utilizing both primary murine MSC and stromal cell lines (ST2), it was found in vitro that MSCs can actively conduct efferocytosis at low levels (30%). It was also found through efferocytosis by MSCs in vivo that efferocytosis was increased during aging (6%) compared to young mice (2%) (FIG. 8B). The data also indicates that neither the type of apoptotic cell engulfed nor the level of efferocytosis influences the receptor pathway involved. However, these variables strongly impacted the fate of efferocytic MSCs. During low levels of efferocytosis, pathways associated with adhesion, proliferation, and mobility are upregulated, while during high levels of efferocytosis, there is increased transcriptional evidence of cellular senescence (p=0.001035, odds ratio 2.49, combined score 29) and apoptosis (p=0.002101, odds ratio 2.61, combined score 23.43), which was confirmed functionally in efferocytic MSCs.

To determine if enhanced MSC efferocytosis induces senescence, a mouse model of enhanced efferocytosis in MSCs (Bai1×PrxCre) was generated. It was found that these MSC's have double the rate of senescence (40% vs. 20%). As increased MSC senescence is associated with bone loss, it was aimed to decrease efferocytosis in vivo. Through RNA sequencing, it was found that MSCs utilize the phosphatidylserine receptor Axl in the activation of efferocytosis.

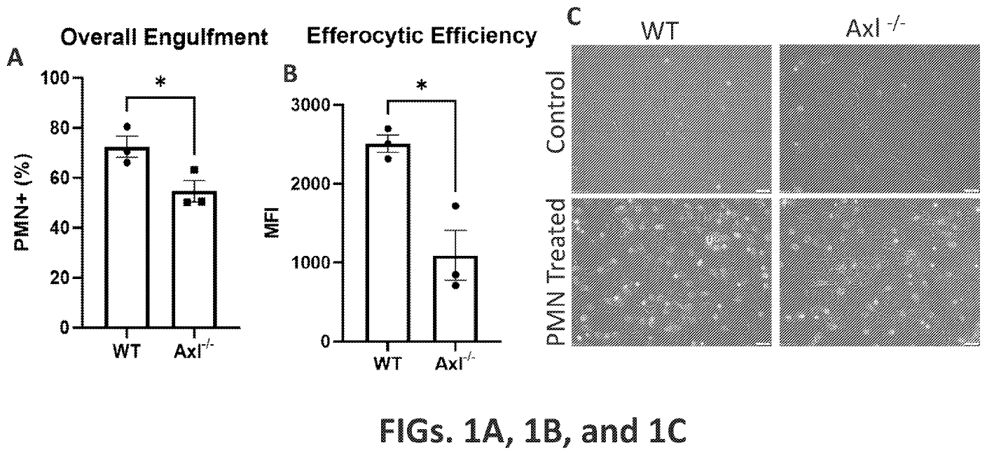

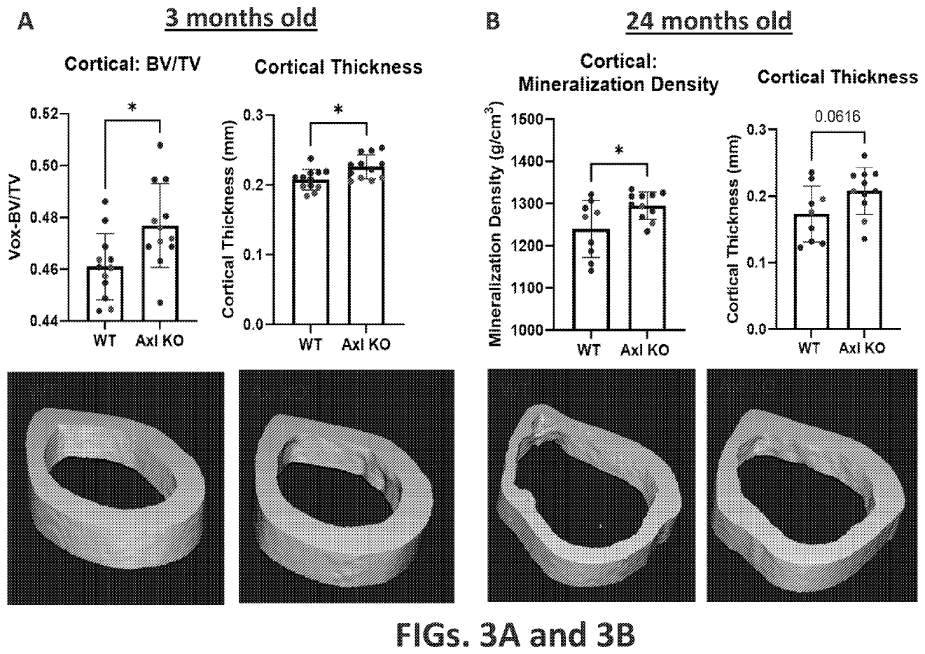

Using a global Axl-mouse model, it was confirmed that loss of Axl decreases the efferocytic efficiency of MSCs to half the control (FIG. 1) and increases both bone mineral density and content (FIGS. 3A and 3B) and biomechanical strength in both young and aged mice. These findings indicate that blocking MSC efferocytosis can improve bone health.

Collectively, these results support the idea that MSCs can utilize established efferocytic pathways but during high levels of efferocytosis, as seen in aging, MSCs become senescent. These data indicate that excessive MSC efferocytosis represents a novel mechanism by which MSCs become senescent and contribute to age-induced bone loss.

### Example 2

### Inhibition of Axl and Tyro3 Blocks Mesenchymal Stromal Cell Efferocytosis and Increases Bone Density in Aged Mice.

Aging induces osteopenia and increases the risk of fractures and the associated morbidity and mortality. The aging mechanisms of bone marrow microenvironment components, particularly mesenchymal stromal cells (MSCs), and how they lead to bone loss during aging remain incompletely understood. Studies have shown that MSC senescence contributes to age-dependent bone loss. However, the mechanisms inducing senescence in MSCs during aging remain unclear.

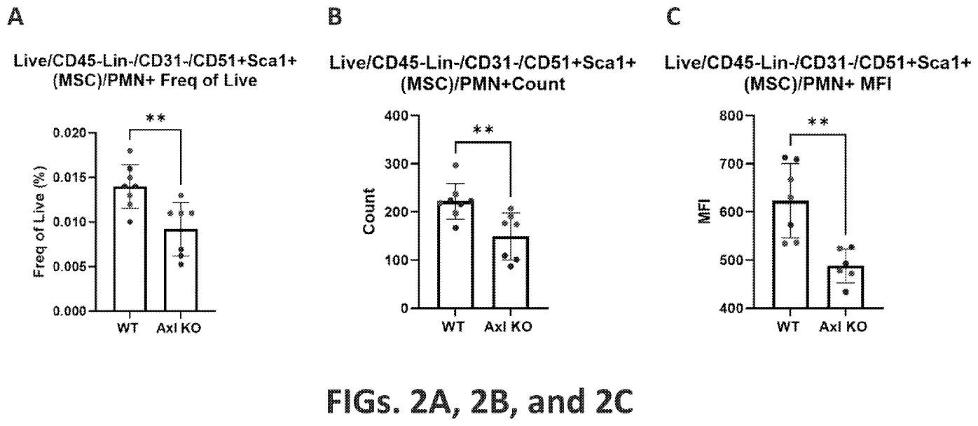

It was demonstrated that aged bone marrow-derived macrophages are deficient in their ability to phagocytose dead cells (efferocytosis) during aging. While macrophages are the primary phagocytes in the bone marrow, MSCs act as non-professional phagocytes, which increases in vivo in aged mice (6% vs. 2%) (FIG. 8B). Using RNA sequencing, the main receptor pathways (Axl, Tyro3, Igtav, Megf10) regulated in efferocytosis was identified, and it was found that the level of efferocytosis strongly influences the fate of efferocytic MSCs. During low levels of efferocytosis, pathways associated with adhesion, proliferation, and mobility are upregulated, while during high levels of efferocytosis, there is increased transcriptional evidence of cellular senescence (p=0.001035) and apoptosis (p=0.002101), which was confirmed functionally in efferocytic MSCs. Since increased senescence is a mechanism of age-related bone loss, it was aimed to block MSC efferocytosis in vivo. RNA sequencing found that Axl transcriptional levels were significantly higher than the other receptors and increased after efferocytosis, indicating that Axl is the principal receptor mediating efferocytosis in MSCs. In mice lacking Axl (Axl^{-/-}), the efferocytic activity of MSCs was decreased (FIG. 1 and FIGS. 2A-C), while bone mineral density (FIGS. 3A and 3B) and biomechanical strength were increased in both young and aged mice. These data indicate that loss of Axl is beneficial for bone health. Surprisingly, the efferocytic rate of MSC's in Axl^{-/-} was reduced but not completely blocked, indicating that, in the absence of Axl, other efferocytic receptors may compensate.

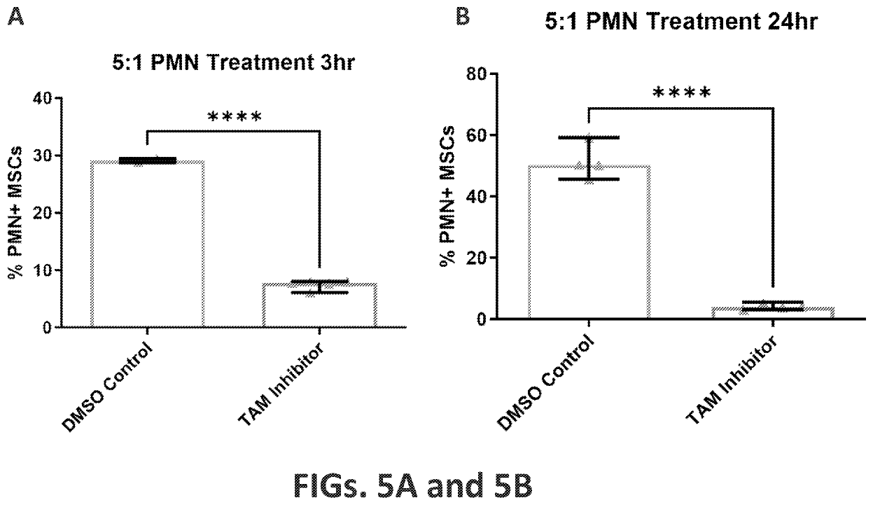

Based on these data, and to determine the translation potential of inhibition of MSC efferocytosis, the efficacy of small molecule inhibitors of TAM receptors (Tyro3, Axl, MerTK) on MSC efferocytosis was assessed in vitro by testing a pan-TAM inhibitor, LDC1267. Initial studies in ST2 cells (stromal cell line) showed that at 24 hours, global TAM inhibition completely blocked MSC efferocytosis without changing cell viability (FIGS. 5A and 5B).

Collectively, the results support the idea that the TAM receptors are critical mediators of MSC efferocytosis and that during high levels of efferocytosis MSCs become senescent. It also demonstrated a targetable mechanism of MSC efferocytosis that may have novel clinical significance in the treatment of age-related bone loss and other diseases caused, at least in part, by efferocytic excess.

### Example 3

### Mesenchymal Stromal Cell Efferocytosis is Blocked In Vitro by Inhibition of Tyro3, Axl, and MerTK Receptors

Bone marrow-derived mesenchymal stromal cells (MSCs) are cells with multi-lineage differentiation capacity that play an important role in the bone marrow niche. While macrophages are the primary phagocytes in the bone marrow, MSCs act as non-professional phagocytes. When MSCs clear high levels of dead and apoptotic cells, a process known as efferocytosis, MSCs have increased senescence. Since increased senescence is a mechanism of age-related bone loss, it was aimed to block MSC efferocytosis. RNA sequencing data showed that MSCs express Axl and Tyro3, receptor tyrosine kinases that mediate macrophage efferocytosis. Importantly, Axl transcriptional levels were significantly higher than Tyro 3 and increased after efferocytosis, indicating that Axl is the principal receptor mediating efferocytosis in MSCs. However, in mice with global deletion of Axl, MSC efferocytosis was reduced but not completely blocked (FIG. 1 and FIGS. 2A-C), suggesting that, while Axl is likely the dominant efferocytosis receptor in MSCs, other receptors may also be involved or may compensate in the setting of Axl loss.

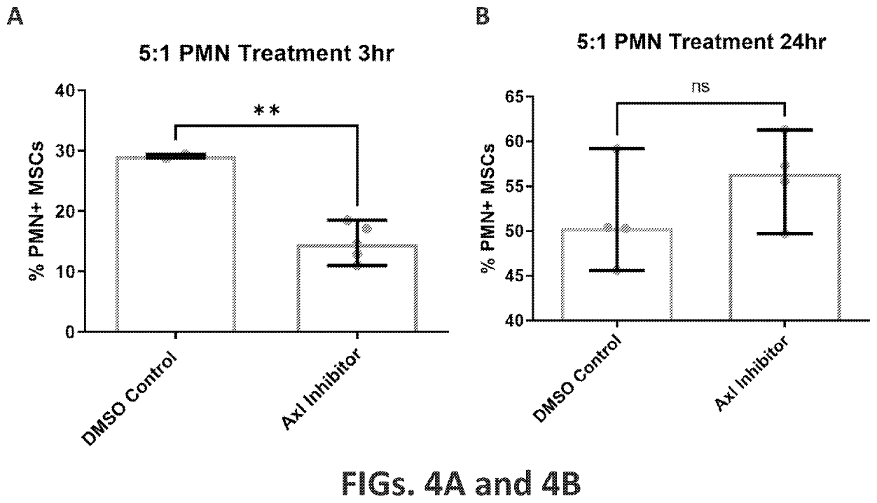

Based on these data, and to determine the translation potential of inhibition of MSC efferocytosis, we assessed the efficacy of small molecule inhibitors of TAM receptors on MSC efferocytosis in vitro by testing a specific Axl inhibitor, TP0903, and a pan-TAM inhibitor, LDC1267. Initial studies in ST2 cells (stromal cell line) and primary human MSCs showed low toxicity with both drugs. Flow cytometry analysis of efferocytic assays showed that, 3 hours after neutrophil addition, neutrophil uptake by MSCs was significantly reduced by both drugs. At 24 hours, global TAM inhibition blocked MSC efferocytosis without changing cell viability (FIGS. 4A-B and FIGS. 5A-B).

These results that inhibition of the TAM receptors reduces MSC efferocytosis in vitro, and identify compensatory TAM dynamics that may account for the partial loss of MSC efferocytosis in mice lacking Axl. Thus, MSC efferocytosis is a targetable mechanism having clinical significance in the treatment of age-related bone loss and other diseases caused, at least in part, by efferocytic excess.

### Example 4

This example describes the materials and methods used in Example 5.

### In Vitro Efferocytosis Assays

ST2 cells, a bone marrow-derived mesenchymal stromal cell line, were plated at 2×10⁴ per cm² in αMEM without ascorbic acid (Gibco)+10% FBS+1% pen-strep and incubated in 21% oxygen at 37° C. until 80% confluency. Neutrophils were isolated from bone marrow of young (8-12 weeks) C57BL/6 mice using the EasySep™ Mouse Neutrophil Enrichment Kit (Stem Cell Technologies) as we previously published (Frisch B J et al. JCI Insight. 2019; 4: e124213) and incubated in RPMI+10% FBS+10 mM HEPES overnight (18-20 h) at 37° C./5% CO₂ to force cells to become end-stage (a.k.a. exhausted) neutrophils as previously described (Casanova-Acebes M et al. Cell 2013; 153:1025-35). End-stage neutrophils were washed with PBS and fluorescently labeled with 20 nM efluro670 (ThermoFischer) according to manufacturer's instructions. Targets (neutrophils or apoptotic lymphocytes) were given in excess (1 MSC: 10 Target) to plated MSCs and incubated for up to 24 h. Cells were then washed 3× with PBS, imaged, and collected for flow cytometry analysis.

### Confocal Microscopy

ST2 cells were infected with a lentivirus to ubiquitously express mCherry. The pLVX-EF1a-IRES-mCherry vector (Clontech) contains an EF1a promoter to constitutively express IRES-mCherry in the infected cells. The detailed methods for generating lentiviral particles, and infecting cells are described as previously published (Ashton J M, et al. Cell Stem Cell. 2012; 11:359-72). The construct was co-transfected with pPax2 (provides packaging proteins) and pMD2.G (provides vesicular stomatitis virus-g envelope protein) plasmids into 293TN (System Bioscience) cells to produce lentiviral particles that were used to infect ST2 cell lines. After expanding the cells after infection, mCherry-positive ST2 cells were sorted on FACS Aria cell sorter (BD Bioscience) for subsequent experiments. Following successful infection, cells were plated at 2×10⁴ per cm² in ascorbic acid-free αMEM+10% FBS+1% pen-strep and incubated at 21% O₂/5% CO₂ at 37° C. until 80% confluent. Neutrophils were isolated from bone marrow of young (8-12 weeks) C57BL/6 UBC-GFP mice using the EasySep™ Mouse Neutrophil Enrichment Kit (Stem Cell Technologies) and incubated in RPMI+10% FBS+10 mM HEPES over-night (18-20 h) at 37° C./5% CO₂ as previously described (Casanova-Acebes M et al. Cell 2013; 153:1025-35)). End-stage neutrophils were then given at a 1:1 ratio to plated MSCs in hypoxia (5% O₂/5% CO₂) at 37° C. for 24 h. Cells were visualized using an inverted Nikon Ti2-E microscope at room temperature using an air-plan apochromat VC×20/0.75 objective. NIS-Elements C with JOBS Acquisition Module software was used to acquire and analyze all images. Work was supported by the Wilmot Cancer Center Imaging and Radiation Shared Resource.

### RNA Sequencing of Murine MSCs

Following sacrifice, soft tissue was removed from the bilateral tibiae, femora, and pelvic bones, and bones were each cut into 3-4 pieces. Bone pieces were crushed with a mortar and pestle to release bone marrow (BM) into PBS+2% FBS. Bone marrow was passed through a 16 G needle to disassociate clumps and pelleted by centrifugation of 1200 RPM for 5 min. Red blood cells were removed via incubation in RBC lysis buffer (156 mM NH₄Cl, 127 μM EDTA, and 12 mM NaHC₃) for 5 min. BM was digested in HBSS containing collagenase type IV (1 mg/mL; Sigma), dispase (1 mg/mL, Gibco), and DNase (10 units/mL, New England Biolabs) for 35 min at 37° C. Digested BM was filtered through a 100 μM cell strainer (Corning) and washed with PBS+2% FBS. Cell numbers were determined using the TC20 Automated Cell Counter (Biorad) and Trypan Blue (Sigma-Aldrich) to exclude dead cells. A two-step approach was used to remove hematopoietic cells, first via magnetic depletion and second via fluorescence-activated cell sorting (FACS).

For magnetic-depletion of hematopoietic populations, BM was labeled with biotinylated antibodies against CD45 and lineage markers (Ter119, B220, CD3e, and Gr1) followed by secondary labeling with streptavidin-conjugated magnetic particles (IMag Streptavidin Particles Plus-DM, BD Biosciences). BM was incubated on the BD IMagnet to magnetically separate CD45+ and lineage+ hematopoietic cells from the non-hematopoietic fraction enriched for stromal cells. The stromal cell-enriched fraction was then labeled with PE-CF594 streptavidin, PerCP-Cy5.5 lineage antibodies, APC-Cy7 CD45, FITC CD31, and PE CD51. Cells were labeled with DAPI to exclude dead cells and FACS-purified using a FACSAria II (BD Biosciences) to remove residual hematopoietic cells (lineage+ and/or CD45+) and endothelial cells (CD31+) to obtain lineage-CD45−CD31−CD51+ marrow stromal cells. Sorted marrow stromal cells were seeded in 12-well plates at 1000 cells/cm² in αMEM (ascorbic acid-free)+10% FBS+1% P/S and incubated in 2% O₂/5% CO₂/37° C. Media was changed on day 4 of culture initiation and every 3-4 days thereafter. Upon reaching confluence, cells were passaged and expanded in 6-well plates. For passaging, cultures were washed with PBS and treated with TrypLE Express (ThermoFisher Scientific) to detach cells. An equivalent volume of culture media was added, and cells were replated at ratios ranging from 1:5 to 1:10. Marrow stromal cells were used at passage 2 or 3 for experiments. In all stromal cultures, flow cytometry was used to assess expression of hematopoietic and macrophage markers (lineage, CD45, CD11b, F4/80), endothelial markers (CD31), and stromal markers (CD51, Sca1, CD140a) and confirmed lack of contamination with macrophages and endothelial cells (data not shown).

Marrow stromal cultures were grown in 6-well plates prepared as described above. Each well was pre-treated with 1 mL of media containing 5×10⁶ apoptotic thymocytes/well. Primary murine apoptotic thymocytes were isolated and prepared as previously published (Chekeni F B, et al. Nature 2010; 467:863-7), fluorescently labeled with 20 nM efluro670 (ThermFischer) according to manufacturer's instructions. Culture plates were centrifuged for 40 s at 100× g and incubated in 5% O₂/5% CO₂/37° C. for 24 h. Control cultures received no target. Stromal cells were washed 3-6× with PBS to remove non-engulfed phagocytic targets. Treated and control cells were then washed 3× with PBS and then collected by FACS-isolated directly in RLT Plus buffer (Qiagen). Both populations were sorted, and control as well as target+ cells were isolated. RNA extraction was performed with Qiagen RNeasy PLUS Micro kit following standard operating procedures of the URMC Genomic Core. RNA quality was assessed using Agilent Bioanalyzer 2100. One nanogram of high-quality (RNA integrity number>8.0) total RNA from each sample was reverse-transcribed into cDNA using the Clontech SMART-Seq v4 Ultra Low Input RNA Kit. Final Illumina libraries were constructed using 200 ng of cDNA with the Illumina 2500HiSeq Library Preparation Kit. All data was analyzed using R version 4.1.0 with Benjamini-Hochberg corrections and packages for DESeq2 (version 1.32.0) with LFC shrinkage software ashr (version 2.2-47), GO.db (version 3.13.0), and EnrichR (version 3.0) with a gene alignment set KEGG_2019 mouse. The GEO dataset can be accessed at https://www.ncbi.nlm.nih.gov/projects/geo/query/acc.cgi?acc-GSE223283. Pathway analysis was performed separately on upregulated and downregulated significantly differentially expressed genes (DEGs) with an adjP<0.05, a baseMean cutoff>100 read counts, and no log fold change cutoff. Gene set enrichment analysis (GSEA) was performed on all genes above a base Mean cutoff>100 read counts without curation for individual gene significance, direction, or fold change.

### RNA Sequencing of ST2 Cells

ST2 cells were plated at 2×10⁴ per cm² in αMEM without ascorbic acid (Gibco)+10% FBS+1% pen-strep and incubated in normoxia at 37° C. until 80% confluent. Neutrophils were isolated from human peripheral blood via Mono-Poly resolving medium (MP Biomedicals, Inc) according to manufacturer's instructions and incubated at −80° C. in FBS+10% DMSO for a minimum of 18 h. End-stage neutrophils were washed with PBS and fluorescently labeled with 20 μM eFluor670 dye (eBioscience) in PBS at 37° C. for 20 min and then washed with RPMI+10% FBS+10 mM HEPES to bind free dye. End-stage neutrophils were then given in excess (10:1) to plated MSCs for 3 or 24 h. Cells were then washed 3× with PBS and collected for isolation via sorting flow cytometry analysis (BD FACSAriaII). ST2 cells and isolated neutrophils were FACS-isolated directly in RLT Plus buffer (Qiagen). RNA extraction was performed with Qiagen RNeasy PLUS Micro kit following standard operating procedures of the URMC Genomic Core. RNA quality was assessed using Agilent Bioanalyzer 2100. One nanogram of high-quality (RNA integrity number>8.0) total RNA from each sample was reverse-transcribed into cDNA using the Clontech SMART-Seq v4 Ultra Low Input RNA Kit. Final Illumina libraries were constructed using 150 μg of cDNA with the Illumina Nextera XT DNA Library Preparation Kit. Differential gene expression was analyzed using R version 4.1.0 using DESeq2 (version 1.32.0) with Benjamini-Hochberg correction and LFC shrinkage software ashr (version 2.2-47). Gene set enrichment (Kolmogorov-Smirnov) and pathway analysis (hypergeometric test) were assessed using EnrichR (version 3.0), and ClusterProfiler (version 3.13) with databases GO.db (version 3.13.0) and KEGG_2019_mouse. The GEO dataset can be accessed at https://www.ncbi.nlm.nih.gov/projects/geo/query/acc.cgi?acc=GSE223283. Pathway analysis was performed separately on upregulated and downregulated significantly differentially expressed genes (DEGs) with an adjP<0.05, a baseMean cutoff>100 read counts, and no log fold change cutoff. Gene set enrichment analysis (GSEA) was performed on all genes above a base Mean cutoff>100 read counts without curation for individual gene significance, direction, or fold change.

### Bioenergetic Profiling

Oxygen consumption rate (OCR) and extracellular acidification rate (ECAR) were measured using Seahorse XF96 (Seahorse Bioscience). Cells were plated on Seahorse 96-well plates 24 h before the experiment at a density of 3×10³ cells/well. Immediately before the experiment, media was replaced with DMEM-XFmedia containing 5 mM glucose, 1 mM glutamine, 1% serum, and no pyruvate. A baseline measurement of OCR and ECAR was taken, and then an inhibitory analysis was performed using injections of oligomycin (Olig) at 1 μM, FCCP at 0.5 μM, and antimycin A (AntA) at 1 μM. After analysis, cells were trypsinized and counted. The following OxPhos and glycolytic indexes were calculated: basal respiration (OCRpre-Olig-OCRpost-AntA), ATP-linked respiration (OCRpre-Olig-OCRpost-Olig), maximal respiration (OCRpost-FCCP-OCRpost-AntA), respiratory capacity (OCRpost-FCCP-OCRpre-Olig), proton leak (OCRpost-Olig-OCRpost-AntA), basic glycolysis (ECARpre-Olig), glycolytic capacity (ECARpost-Olig), and glycolytic reserve (ECARpost-Olig-ECARpre-Olig). ATP was measured using the CellTiter-Glo kit (Roche).

### Measuring Mitochondrial Networking

Human MSCs (hMSC) cells were seeded on laminin coated 25 mm glass coverslips that are placed inside of wells on a 6-well plate and incubated at 5% O₂/5% CO₂/37° C. hMSCs were exposed to end-stage neutrophils (described above) stained with calcein AM (ThermoFisher) at 1 μM. hMSCs were then incubated in HBSS containing mitochondria-specific fluorescent probe, MitoTracker Red (ThermoFisher), at 100 nM to visualize mitochon-dria. hMSCs were visualized using the AxioVert microscope and ×40 magnification. Images were captured and analyzed in ImageJ using Mitochondrial Network Analysis (MiNA) plugin for ImageJ. ImageJ Convolve feature was applied, allowing for the stained mitochondria to be highlighted. The threshold feature highlights mitochondria, minimizing the background. In the process of analyzing, only mitochondria>25 pixels were selected to further minimize background noise (Yu T, et al. Cardiovasc Res. 2008; 79:341-51).

### Measuring Mitochondrial Membrane Potential

Human MSCs (hMSC) were exposed to end-stage neutrophils (described above) and stained with calcein AM (ThermoFisher) at 1 μM. After washing, hMSCs were incubated in phenol red-free media containing membrane potential (ΔΨm) sensitive probe Tetramethylrhodamine ester, TMRE (ThermoFisher), at 20 nM for 30 min at 37° C. In parallel, a set of cells undergoing efferocytosis was stained with Nonyl Acridine Orange (NAO, ThermoFisher) at 100 nM to detect possible changes in mitochondrial mass. Cells were then lifted from plates and analyzed using an 18-color LSR Fortessa flow cytometer (BD Biosciences). DAPI was present in the assay media to gate out dead cells. DAPI⁻ (viable)/calcein⁺ (efferocytic)/TMRE⁺ cells were analyzed for TMRE signal to measure ΔΨm in efferocytic cells. As a negative control, cells were added with antimycin A at 1 μM to depolarize mitochondria. A difference in TMRE signal between polarized and depolarized mitochondria was taken as a measure of ΔΨm. In parallel, DAPI (viable)/calcein (efferocytic)/NAO cells were analyzed for NAO signal to measure mitochondrial mass. TMRE signal was normalized to NAO signal to account for possible changes in mitochondrial mass.

### Differentiation Assays

ST2 cells were plated at 2×10⁴ per cm² in αMEM without ascorbic acid (Gibco)+10% FBS+1% pen-strep and incubated in 5% CO₂/37° C. until 80% confluent. Neutrophils were isolated from bone marrow of young (8-12 weeks) C57BL/6 mice using the EasySep™ Mouse Neutrophil Enrichment Kit (Stem Cell Technologies) and incubated in RPMI+10% FBS+10 mM HEPES overnight (18-20 h) at 5% CO₂/37° C. as previously described (Casanova-Acebes M, et al. Cell 2013; 153:1025-35). End-stage neutrophils were washed with PBS and fluorescently labeled with 20 nM efluro670 (ThermoFischer) according to manufacturer's instructions. Targets were then given at baseline (1:1) and in excess (1:2 and 1:3) to plated MSCs for 24 h. Following incubation, cells were washed 3× with PBS, imaged, and given supplemented media every 2-3 days for 21 days to differentiate down osteoblastic (αMEM with ascorbic acid (Gibco)+10% FBS+1% pen-strep+10 mM β-glycerolphosphate+50 μg/mL ascorbic acid) or adipocytic lineage (α-MEM media without ascorbic acid (Gibco)+10% FBS+1% pen-strep+5000 nM insulin+100 nM dexamethasone) or sorted for PMN+/− cells alongside controls and replated at 2× 10⁴ per cm² for 24 h before being given supplemented media as described above. Cells were stained for alkaline phosphatase and von Kossa for mineralization formation or with BODIPY for lipid formation every 7 days for 21 total days.

### BODIPY Staining

Cells were washed with PBS 3×, then stained with 10 mM BODIPY (Invitrogen™) used at 1:1000 ratio in media (α-MEM media+10% FBS+1% pen-strep) for 30 min at room temperature (RT) in the dark. Following the incubation, the cells were washed 3× with PBS and imaged via light microscopy. Images were then quantified for adipocyte formation using ImageJ software.

### Alkaline Phosphatase and Von Kossa Staining

Cells were washed with PBS 3× and then fixed with formalin for 30 min at room temperature (RT). Following fixation, the cells were washed with water 3× and left to sit for 15 min at RT while preparing alkaline phosphatase (AP) staining. To prepare the AP stain, 5 mg Naphthol AS MX-PO4 was dissolved in 200 μL of N,N-dimethylformamide (DMF), 25 mL 0.2 M Tris pH 8.3, and 25 mL water. Red Violet LB salt (30 mg) was added to solution, vortexed, and filter through 45 μm filter. After incubation with AP stain for 45 min RT, cells were washed with water 3× and then stained with 2.5% silver nitrate for 30 min RT in a ventilated area, then washed with water and air dried. All plates were imaged via light microscopy and quantified for osteoblastic formation using ImageJ software.

### Light Microscopy

Images were also taken at room temperature on an Olympus BX41, Olympus DP70 camera, and 20× and 4× UPlanFl objective (NA 0.5). CellSense software (Olympus) was used to acquire images. All images captured in bright field and with filters (FITC) were overlaid using ImageJ software.

Efferocytosis Assays with Pharmacological Inhibition of Mitochondrial Fission

Primary human MSCs (hMSCs) cells (Lonza) were plated at 2×10⁴ per cm² in αMEM+10% FBS+1% pen-strep and incubated in 5% O₂/5% CO₂/37° C. until 80% confluent. Neutrophils were isolated from human peripheral blood via Mono-Poly resolving medium (MP Biomedicals, Inc) according to manufacturer's instructions and incubated in RPMI+10% FBS+10 mM HEPES overnight (18-20 h) at 37° C./5% CO₂ as previously described (Casanova-Acebes M, et al. Cell 2013; 153:1025-35). hMSCs were treated with 25 μM Mdivi for 1 h prior to giving 20 nM efluro670 (ThermoFischer) stained end-stage neutrophils in excess (1:10) for 24 h. Cells were then washed 3× with PBS, imaged, and collected for flow cytometry analysis.

Alkaline Phosphatase Measurements with Pharmacological Inhibition of Mitochondrial Fission

Cells that performed efferocytosis in the presence or absence of Mdivi, were washed and stained for AP as described above. Wells were photographed, and images were analyzed using ImageJ for staining intensity.

### Flow Cytometry

All samples were run on a LSRII flow cytometer: 3 lasers, 355 nm, 488 nm, and 633 nm (BD Biosciences). Analysis was performed using FlowJo version 10.8 (Tree Star). Sorting was done on a FACSAria II with 405-, 488-, 532-, and 633 nm lasers (BD Biosciences). All flow cytometry equipment is housed and quality controlled within the URMC Flow Cytometry core.

### Statistics

All data are presented as mean±SD. All analyses were performed with GraphPad Prism software (version 9.2.0) using two-tailed Student's/test, 1-way or 2-way ANOVA with Tukey's multiple-comparisons post-test as appropriate. A p value<0.05 was considered significant, and any values nearing significance were stated exactly.

### Example 5

### Mesenchymal Stromal Cells (MSCs) Participate in Efferocytosis

Previous studies have noted that MSCs can phagocytose bacteria, metallic particles from prosthetics, collagen, and apoptotic cells. However, dynamics and impact of this MSC activity remain poorly understood. Billions of cells return to the bone marrow to be cleared daily by phagocytes, with a large component being neutrophils (up to 60%), making them a likely efferocytic target for professional phagocytes, such as macrophages, and non-professional phagocytes, such as MSCs. To investigate the impact of efferocytosis on MSC, a flow cytometric analysis of neutrophil uptake by ST2 cells, a murine bone marrow-derived mesenchymal stromal cell line, was first performed. The assay showed that ST2 cells conduct efferocytosis of end-stage murine neutrophils (PMNs). Through microscopy, it was confirmed that ST2 actively engulfed end-stage PMNs, as evidenced by the void left in the cytoplasm and z-stack imaging. Taken together, these data confirm that MSCs can actively participate in efferocytosis. However, the impact on MSCs ability to support normal function following efferocytosis remains to be elucidated.

In general, non-professional phagocytes have a limited efferocytic capacity in comparison to professional phagocytes, as they do not possess as many phagocytic receptors or produce reactive oxygen species (ROS) as readily to assist with degradation of internalized targets. To define the efferocytic machinery utilized by MSCs, RNA sequencing was conducted on ST2 cells exposed to excess human PMNs (1:10). Cells were then harvested at 3 and 24 h after the addition of PMNs and separated by fluorescence-activated cell sorting (FACS) based on presence of the target label. At quality control (QC) check via bioanalyzer, RNA from human PMN targets was highly degraded and gave insufficient RNA quantity and RNA integrity number (RIN). Thus, similar PMN time point samples were pooled (Supplemental Table 1). To ensure that the transcripts seen were those of the efferocytic cell (ST2) and not the target (hPMNs), the dataset was probed for genes unique to the target (e.g., Ptprc, Itgam, Itgax, L-selectin), and there was no evidence (i.e., average read count<3) suggesting RNA contamination from the targets.

Principal Component Analysis showed that the genetic profiles of early (3 h) and late (24 h) stage efferocytic cells differ greatly from each other and from that of non-efferocytic (control) cells. As expected, based on their functional ability to perform efferocytosis, MSCs express the transcripts for numerous phagocytic and efferocytic receptors and signaling pathways even before efferocytic challenge. Notably, phagocytic and efferocytic receptors are upregulated at 3 h post efferocytosis (Axl, Tyro3, Itagy, etc.), while transcripts of molecules required for internal processing pathways necessary to degrade apoptotic cargo (e.g., Elmo1, Elmo2, Dock1, Gulp1) are upregulated at 24 h. Notably, MerTK, the principal receptor for efferocytosis by bone marrow macrophages, is not expressed in ST2 cells (normalized read count 25±10). Therefore, MSCs demonstrate dynamic expression of key molecules in the efferocytic machinery in response to efferocytosis and a collective efferocytic machinery profile distinct from professional phagocytes within the bone marrow compartment.

While end-stage neutrophils are the most likely target in the bone marrow, whether MSCs are capable of engulfing other types of apoptotic cargo was to be determined. It was found that bone marrow-derived primary murine MSCs can also engulf apoptotic thymocytes. The transcriptional analysis of primary murine bone marrow-derived stromal cells exposed to apoptotic thymocytes (SCAT) identified the same efferocytic pathways seen in ST2 cells, including a similar lack of MerTK expression (normalized read count 28±6). These data identify the key efferocytic mediators in primary murine MSCs and indicate that similar machinery is used regardless of apoptotic target.

### Efferocytosis by MSCs Induces Stress Response

A gene set enrichment analysis (GSEA) and pathway analysis using the Kyoto Encyclopedia of Genes and Genomes (KEGG) database to predict the functional impact of efferocytosis on MSCs. Consistent with efferocytic behavior, there was transcriptional evidence of increased genes involved in the phagosome and lysosome. In addition, this analysis identified evidence of global MSC stress through transcriptionally downregulated metabolic and biogenesis pathways along with upregulated genes involved in cellular senescence and apoptosis. Notably, these changes occurred in both efferocytic ST2 cells and primary murine MSCs regardless of type of apoptotic cargo. To functionally confirm the increased cellular senescence identified by pathway analysis, beta-galactosidase (β-Gal) and proliferative potential were measured in efferocytic ST2 cells. β-Gal activity was increased in efferocytic (PMN+) ST2 cells compared to non-efferocytic (PMN−) ST2 cells. Consistent with increased cellular senescence, sorted PMN+ST2 cells had decreased cell replication. Therefore, the data indicate that efferocytosis induces significant cellular stress for MSCs.

### Mesenchymal Stromal Cell Efferocytosis Disrupts Osteoblastic and Adipocytic Differentiation Capacity

To test the impact of efferocytosis on MSC differentiation, osteoblastic and adipocytic differentiation in MSCs was induced following a challenge with an efferocytic target. ST2 cells were exposed to PMNs in a ST2:PMN ratio of 1:1, 1:2 and 1:3 for 24 h, and after removing non-engulfed PMNs, the ST2 cells were exposed to osteoinductive or adipo-inductive media. Even though only a subset of ST2 cells were PMN+ at 24 h in prior experiments, exposure of ST2 cells to PMNs decreased osteoblastic differentiation as measured by alkaline phosphatase and Von Kossa staining in comparison to ST2 cells that were not exposed to PMNs.

To determine if the decreased alkaline phosphatase activity was a result of cell-autonomous efferocytic activity, PMN+ and PMN-ST2 cells were separated by FACS. It was found that efferocytic MSCs (PMN+) had decreased alkaline phosphatase staining, while their non-efferocytic (PMN−) counterparts surprisingly had a small but significant increase in alkaline phosphatase staining in comparison to the controls, which were also subjected to the FACS fluidics without separation. Therefore, efferocytosis by MSCs induces cell-autonomous defects in MSC osteoblastic differentiation. Consistent with the functional defects in osteogenesis, positive osteogenic regulator genes, such as Osr1, Bmp4, Omd, and Igf-1, are decreased, while negative osteogenic regulator genes, such as Suv39h1, are increased following efferocytosis. Similar to the inhibition in osteogenesis, efferocytic MSCs have decreased adipocytic differentiation, as shown by decreased lipid vacuole formation following induction. Consistent with the functional defects in adipogenesis, positive adipogenic regulator genes such as Cebpa, Cebpg, Srebf1, and Fosb are decreased following efferocytosis. Taken together, these data indicate that efferocytosis disrupts the differentiation capacity of MSCs by restraining MSC differentiation rather than by shifting from osteoblastic to adipocytic cell differentiation.

### Efferocytosis by MSCs Disrupts Metabolism and Mitochondrial Networks

Many of the pathways identified in the transcriptional program of efferocytic MSCs relate to dysregulation of metabolism. To test whether MSC efferocytosis induced metabolic disruption, the oxygen consumption rates (OCR) and extracellular acidification rates (ECR) were measured, which are measurements for oxidative phosphorylation and glycolysis, respectively. Consistent with the transcriptional data, efferocytosis by MSCs decreases oxidative phosphorylation and glycolysis. To determine if the decreases in oxidative phosphorylation and glycolysis were a result of environmental stress or cell-autonomous efferocytic activity in MSCs, PMN+ and PMN-MSCs were sorted, and OCR and ECR were measured. It was found that the most drastic metabolic disruption was present in efferocytic MSCs (PMN+) for both oxygen consumption and glycolysis, while non-efferocytic (PMN−) MSCs had relatively preserved glycolysis. These data demonstrate that efferocytosis significantly alters metabolic processes in MSCs.

### Inhibition of Mitochondrial Fission Decreases Efferocytosis and Rescues Osteoblastic Differentiation Impairment

Since oxidative phosphorylation was impacted more heavily than glycolysis in a cell-autonomous fashion, it was hypothesized that efferocytosis by MSCs may lead to mitochondrial remodeling. Using the ST2 RNA sequencing dataset, mitochondrial dynamics genes and pathways were probed, and efferocytosis-mediated dynamic regulation of genes associated with mitochondria fission and fusion was observed. Early in the efferocytic process (3 h), MSCs upregulate fusion genes, such as Mfn2 and Opa1. However, as efferocytosis progresses (24 h), MSCs upregulate fission genes, such as Fis1 and Dmnl1. These data indicate that MSCs, after performing efferocytosis, shift from a state of mitochondrial fusion to fission. To confirm mitochondrial remodeling in efferocytic MSCs, mitochondria was labelled in control and PMN+ MSCs with MitoTracker Red or TMRE to assess the mitochondrial length and membrane potential, respectively. It was found that both the mitochondrial length and TMRE signal were decreased in efferocytic MSCs, consistent with mitochondria undergoing fission. Together, these data show that, as a result of efferocytosis, MSCs have diminished oxidative function and remodeled mitochondria, consistent with mitochondria undergoing fission.

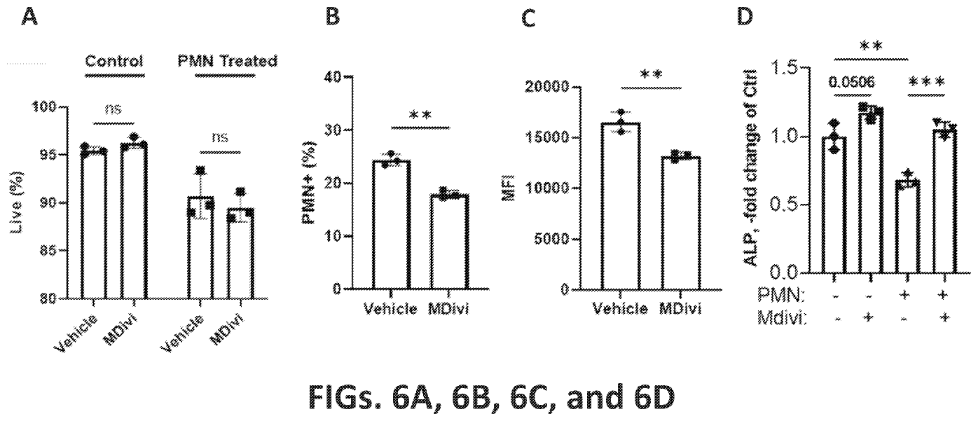

Mitochondrial metabolism plays a key role in MSC's ability to support tri-lineage differentiation, whereby disruption of homeostatic metabolism can impact osteoblastic differentiation and subsequent bone formation. During normal osteoblastic differentiation, MSCs undergo fusion, or mitochondrial lengthening. Blocking fusion and enforcing fission disrupts MSC differentiation to the osteoblastic lineage. Since the data show that mitochondria undergo fission, or shortening, during MSC efferocytosis, it was hypothesized that efferocytosis-induced mitochondrial fission may mediate the block in osteoblastic differentiation initiated by MSC efferocytosis. Since this is a key metabolic switch that impacts bone health and may represent a mechanism of bone loss and osteoporosis, whether MSCs isolated from human bone marrow are also capable of efferocytosis was tested. Human MSCs (hMSCs) demonstrated a similar rate of efferocytosis as murine MSCs. Similar to its effects on murine MSCs, efferocytosis also inhibited hMSCs differentiation to osteoblasts (FIG. 6). To determine if the decreased osteoblastic differentiation potential is a result of mitochondrial fission, hMSCs were treated with Mdivi, an inhibitor for mitochondrial fission, prior to efferocytosis. The overall rate and efficiency (MFI) of efferocytosis, tested at 24 h, was decreased in hMSCs pre-treated with Mdivi without impacting their viability (FIG. 6). Consistent with the role of mitochondrial remodeling in MSC differentiation to osteoblasts, there was a trending increase in osteoblastic differentiation with Mdivi treatment in the absence of PMN (FIG. 6). Importantly, co-treatment with Mdivi and PMN rescued the osteoblastic differentiation potential of hMSCs (FIG. 6). In summary, inhibiting mitochondrial fission in the setting of MSC efferocytosis rescues osteoblastic differentiation potential in efferocytic MSCs. These data demonstrate that, in MSCs, increased mitochondrial fission mediates the defect in osteoblastic differentiation induced by efferocytosis.

### Discussion

This example demonstrates that engulfment of apoptotic cells by bone marrow-derived MSCs, a previously underreported function of MSCs, disrupts their metabolism and inhibits their differentiation, with potential impact on bone health. In the bone marrow microenvironment (BMME), MSCs carry out numerous functions, including the support of hematopoiesis, immunomodulation, and differentiating down the osteoblastic, adipocytic, and chondrocytic lineages to support bone growth, maintenance, and repair. Previously non-professional or non-specialized phagocytes, cells that have been noted to conduct phagocytosis only under specific circumstances or only eating specific targets, were not identified in the bone marrow. However, MSCs had been reported to contribute to phagocytosis in the embryo. Follow-up studies in vitro confirmed phagocytosis and efferocytosis capacity of MSC. Given the metabolic and mitochondrial impact of efferocytosis in macrophages, MSC efferocytosis may have a metabolic impact, and it may inhibit their differentiation.

The data presented herein show that MSCs can act as a non-professional phagocyte in vitro using ST2 cells and primary BM MSCs from is challenging to isolate RNA even from freshly isolated non-apoptotic PMNs. Unlike other commonly used targets for efferocytic experiments such as apoptotic thymocytes, the data demonstrates that apoptotic PMNs do not contribute significant amounts of RNA to phagocytic populations even at early time points.

Transcriptomic studies of efferocytosis by MSCs found that MSCs upregulate pathways related to phagocytic behaviors, including regulation of actin cytoskeleton, focal adhesion, phagosome, and lysosome, regardless of efferocytic target type. Indeed, MSCs possess the necessary receptors to conduct efferocytosis, with Axl, Tyro3, and Megf10 being the most prominent receptors-pathways regardless of efferocytic target. In response to engulfment of apoptotic targets, MSCs also upregulate internal processing, such as Dock1, Elmo1, Gulp1, and the Axl transcriptional target and accessory protein Gas6, which is necessary to activate a functional response. While Axl and Tyro3 are expressed, MerTK, the third TAM receptor, is not expressed on MSCs. This is a key receptor pathway of professional phagocytes such as macrophages. These data indicate that MSCs act as a supporting phagocyte within the BMME, and they do not rely on MerTK. Since a number of small molecules have been developed to differentially target and inhibit each TAM receptor, it may be possible to selectively inhibit MSC efferocytosis without blocking macrophage activity.

While the most likely primary efferocytic targets for MSCs in the bone marrow are end-stage neutrophils, MSCs are known to conduct phagocytosis of bacteria, metallic particles from prosthetics, and collagen. Although efferocytosis is a specialized form of phagocytosis, and the molecular mechanisms of efferocytosis closely resemble those of phagocytosis.

The data presented here demonstrate that efferocytic activity causes a stress response in MSCs in the form of increased cellular senescence and apoptosis. As cellular senescence has been shown to decrease differentiation capacity, it was hypothesized that MSC efferocytosis would decrease differentiation capacity. Consistent with this, efferocytic MSCs display a diminished capacity to differentiate toward the osteoblastic and adipocytic lineages.

While cellular senescence has many causes, it has been reported to be induced by disrupted metabolic activity in which the mitochondrial dynamics between fission and fusion play a role in energy trafficking in the cell. In addition, phagocytosis by macrophages is accompanied by changes in mitochondrial dynamics (fission) and oxidative function, which in turn promotes further phagocytosis. In contrast, during MSC differentiation, mitochondria increase their fusion. Thus, the dynamics of the mitochondrial networks between efferocytosis (fission) and differentiation (fusion) are opposing. The RNA sequencing data found that efferocytic MSCs display decreased regulation of metabolic pathways and biogenesis genes, leading to a hypothesis that efferocytic activity in MSCs may alter mitochondrial dynamics as a mechanism that decreases their differentiation capacity. Consistent with this, both oxidative phosphorylation and glycolysis, two of the major energy synthesis pathways, are decreased in MSCs following efferocytosis. Transcriptionally, the fission-promoting Fis1 and Dmnl1 genes were upregulated. Decreased mitochondrial length and membrane potential are consistent with an increase in a fission-like state for the mitochondrial network and mitochondrial dysfunction in efferocytic MSCs, indicating that efferocytosis in MSCs impacts mitochondrial remodeling as it does in macrophages. To determine if inhibition of osteoblastic differentiation induced by efferocytosis was caused by mitochondrial remodeling, fission was pharmacologically inhibited using Mdivi. The data shows that inhibition of fission increases osteoblastic differentiation independent of efferocytic activity, and shows that pharmacologic inhibition of fission not only decreases efferocytic activity in MSCs but also rescues the defect in osteoblastic differentiation induced by MSC efferocytosis. These data demonstrate for the first time that efferocytosis impairs MSC differentiation by altering mitochondrial remodeling. While efferocytosis by MSCs may be beneficial to help professional phagocytes in the BMME by serving as a supportive non-professional phagocyte, the data indicates that this may come with detrimental consequences on osteoblastic differentiation.

This example identifies the MSCs' role in the process of removing apoptotic cells from the bone marrow as a previously unappreciated mechanism of MSC dysfunction. Professional phagocytes, such as macrophages, regulate their non-professional counterparts, so that non-professional phagocytic cells are recruited when macrophages are either defective or insufficient to engulf apoptotic cells. MSCs may, therefore, be engaged as non-professional phagocytic cells, especially (or exclusively) when macrophage populations are depleted or dysfunctional. Consistent with this, in an embryological study on mice lacking macrophages due to genetic loss of the pu.1 gene, MSCs gained efferocytic capabilities in vivo. Relevant to the role of MSCs as skeletal precursors, in the absence of c-fms+ cells (early and late macrophages), there was an increase in apoptotic cells in the bone marrow, which was associated, unexpectedly, with a reduction in bone mass and bone formation. In this context, it is possible that the decreased bone mass and bone loss observed may be due to recruitment and increased efferocytosis by MSCs. However, targeting via clodronate unexpectedly did not result in bone loss. The novel role of MSCs as non-professional efferocytic cells may explain this finding. While clodronate is able to target macrophages, it is non-specific and, therefore, may target other phagocytic cells such as neutrophils (the main apoptotic cell population in the bone marrow) and MSCs. In the setting of MSC efferocytosis, uptake of clodronate may protect the BMME by killing the MSC before it becomes senescent upon activation of efferocytic pathways, protecting from bone loss.

Numerous studies have demonstrated diminished macrophage efferocytic potential and increases in apoptotic cell burden in vivo in the setting of aging and diseases such as autoimmunity, obesity and diabetes. For example, significant defects in efferocytosis by bone marrow macrophages were observed in aged mice, indicating that at least some of the MSC senescence observed in aging may be a result of MSC efferocytosis. Thus, enhanced MSC efferocytosis and subsequent MSC dysfunction represents a novel mechanism of dysfunction in bone loss and decreased bone formation associated with these conditions.

Efferocytosis by MSCs may also have important consequences for their roles in the setting of cancer. Tumor-associated macrophages have been shown to promote tumor growth following efferocytosis, which is abundant in tumors, especially in response to cytotoxic therapies, by suppressing tumor immunity and limiting the anti-tumor response. Thus, it is possible that MSCs efferocytosis could lead to an immune-suppressive/pro-tumorogenic microenvironment in bone and bone marrow in response to tumors and their metastases, especially in the setting of cytotoxic therapies.

In summary, efferocytosis by MSCs represents a mechanism of MSC dysfunction and senescence leading to age- and disease-associated bone marrow remodeling and bone loss. Together, these data demonstrate a novel mechanism by which MSC becomes senescent contributes to bone loss, and disrupts the bone marrow microenvironment. This example also identifies pharmacologically targetable mechanisms for MSC efferocytosis that can have clinical significance in the treatment of age- and disease-related bone marrow remodeling and bone loss caused, in part, by excessive MSC efferocytosis.

### Example 6

Age-dependent bone loss is a manifestation of skeletal aging. Hallmarks of skeletal aging include mesenchymal stromal/stem cell (MSC) dysfunction and cellular senescence. However, the mechanisms inducing MSC senescence remain unclear. It was found that MSCs contribute to the clearance of apoptotic cells in the bone marrow (efferocytosis). While rates of efferocytosis are low at homeostasis, in aging, MSCs increase their efferocytic activity (FIG. 8B). Thus, it was hypothesized that excess efferocytosis may contribute to MSC dysfunction and senescence, and represent a previously unknown mechanism of bone loss.

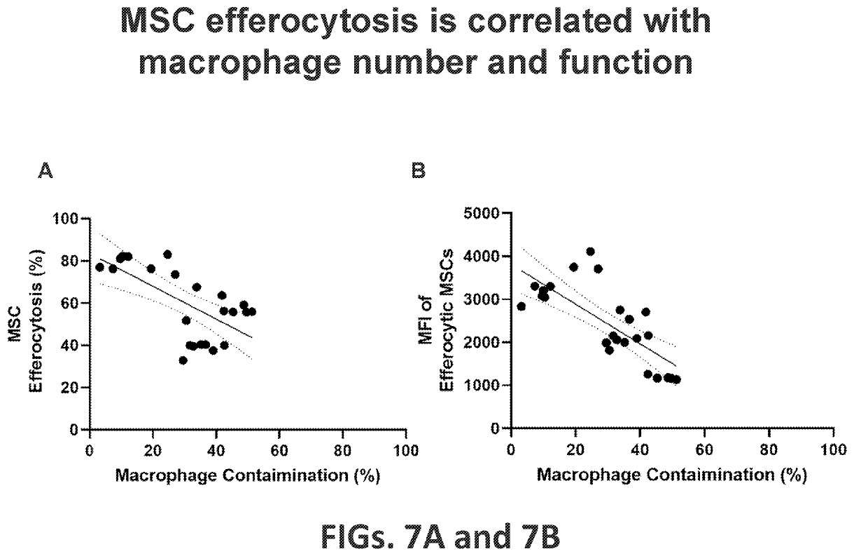

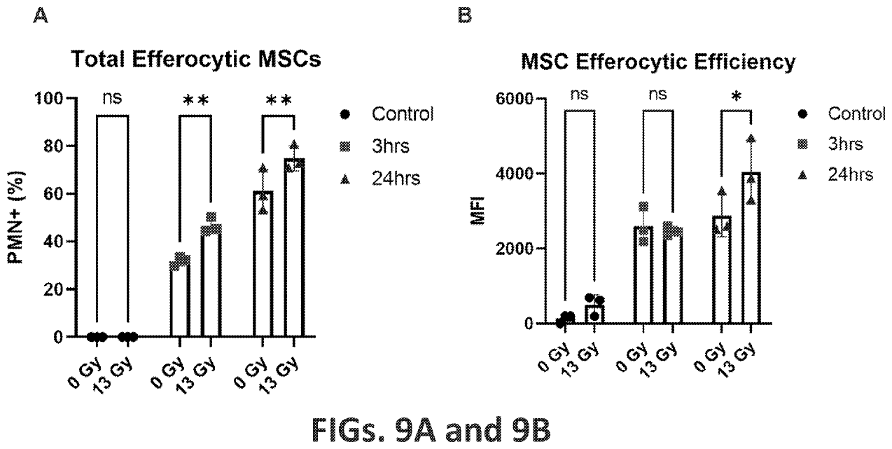

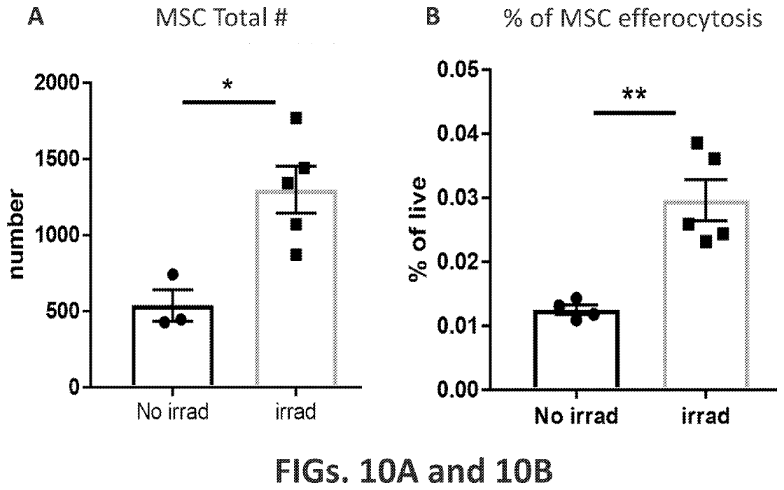

In vitro, excess efferocytosis induced MSC dysfunction through increased mitochondrial fission and metabolic disruption. Additionally, MSCs with high efferocytic burden had transcriptional evidence of increased senescence, which we confirmed functionally. In a mouse model of enhanced efferocytosis by MSCs (Bai1×PrxCre), increased efferocytosis by MSCs (70% to 85%) was observed, and efferocytic MSCs had increased rates of senescence compared to controls (40% vs. 20%). Consistent with a negative effect of excessive efferocytosis on bone homeostasis, Bai1×PrxCre mice at 3 months had decreased cortical thickness, which significantly declined with age (12 m) compared to controls. In this model, MSC dysfunction was also decreased by CFU-F/OB. Next, the efferocytic machinery in MSCs was next profiled, and Axl was identified as the primary efferocytic receptor on MSCs. In mice with global loss of Axl, MSCs had decreased efferocytic efficiency, and increased bone mineral density/content and cortical thickness, in both young (3 m) and aged (24 m) mice (FIGS. 3A-B). To determine the translation potential of this finding, small molecule inhibitors of the TAM receptors (Tyro3, Axl, MerTK) were tested on MSC in vitro and found that combined Axl and Tyro3 inhibition blocks MSC efferocytosis (FIGS. 4A-B and FIGS. 5A-B). Collectively, the data support the idea that excess efferocytosis is a novel mechanism of bone loss. MSC efferocytosis is activated by defects in professional phagocytic cells, such as macrophages (FIGS. 7A-B and FIG. 8A). Therefore, senescence induced by excessive MSC efferocytosis is an underappreciated mechanism of bone loss in settings of defective macrophages, as in aging, obesity, and diabetes-induced bone loss. Given the unique reliance of MSC efferocytosis on Axl, this novel mechanism is pharmacologically targetable for the treatment of bone loss in aging and in other diseases caused, in part, by MSC efferocytic excess, including accidental or therapeutic exposure to ionizing radiation (FIGS. 9A-B and FIGS. 10A-B).

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method of increasing bone density or reducing bone loss or bone resorption in a subject in need thereof, comprising administering to the subject an effective amount of an agent capable of reducing efferocytosis by mesenchymal stromal cells.

2. A method of treating a disease or disorder associated with reduced bone density resorption in a subject in need thereof, comprising administering to the subject an effective amount of an agent capable of reducing efferocytosis by mesenchymal stromal cells.

3. The method of claim 1, wherein the mesenchymal stromal cells comprise bone marrow mesenchymal stromal cells.

4. The method of claim 1, wherein the agent is a small molecule compound, an oligonucleotide, a nucleic acid, a peptide, a polypeptide, or an antibody or an antigen-binding portion thereof.

5. The method of claim 1, wherein the agent comprises an inhibitor of a Tyro3, Axl, and Mer (TAM) receptor kinase or an inhibitor specific to an Axl receptor kinase, or a derivative thereof.

6. The method of claim 5, wherein the agent comprises a pan-TAM inhibitor.

7. The method of claim 1, wherein the agent comprises LDC1267, TP0903, bosutinib, BGB324, crizotinib, foretinib, BMS-777607, LY2801653, amuvatinib, BMS-796302, cabozantinib, MGCD265, NPS-1034, LDC1267, gilteritinib, SGI-7079, TP-0903, UNC2025, S49076, sunitinib, 12A11, Mab173, YW327.6S2, D9, E8, merestinib, ASLAN002, SGI-7079, or a combination thereof.

8. The method of claim 7, wherein the agent comprises LDC1267 or a derivative thereof, and wherein LDC1267 is represented by the following structure:

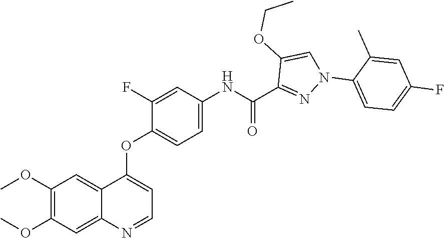 [SMILES: CCOc1cn(-c2ccc(F)cc2C)nc1C(=O)Nc1ccc(Oc2ccnc3cc(OC)c(OC)cc23)c(F)c1]

9. The method of claim 7, wherein the agent comprises TP0903 or a derivative thereof, and wherein TP0903 is represented by the following structure:

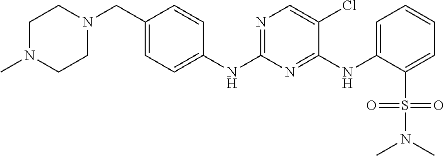 [SMILES: CN1CCN(Cc2ccc(Nc3ncc(Cl)c(Nc4ccccc4S(=O)(=O)N(C)C)n3)cc2)CC1]

10. The method of claim 1, wherein the agent comprises a mitochondrial division inhibitor.

11. The method of claim 10, wherein the agent comprises mitochondrial division inhibitor 1 (Mdivi-1) or a derivative thereof, and wherein Mdivi-1 is represented by the following structure:

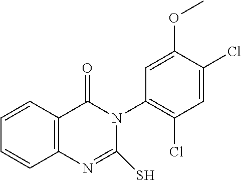 [SMILES: COc1cc(-n2c(S)nc3ccccc3c2=O)c(Cl)cc1Cl]

12. The method of claim 1, wherein treatment by the agent results in 10-15% reduction of bone loss or 10-15% increase of bone density.

13. The method of claim 1, wherein the subject has increased efferocytosis by mesenchymal stromal cells caused by one or more conditions.

14. (canceled)

15. (canceled)

16. (canceled)

17. (canceled)

18. (canceled)

19. The method of claim 2, wherein the disease or disorder associated with reduced bone density is osteoporosis, a critical sized-bone defect, a mechanical disorder resulting from disuse or injury, osteogenesis imperfecta, osteomalacia, bone necrosis, rickets, osteomyelitis, alveolar bone loss, Paget's disease, hypercalcemia, primary hyperparathyroidism, metastatic bone diseases, myeloma, or bone loss.

20. The method of claim 1, wherein the bone loss is caused by aging, cancer, fibrous dysplasia, aplastic bone diseases, metabolic bone diseases, type 1 diabetes, lupus, rheumatoid arthritis, inflammatory bowel disease, hyperthyroidism, celiac disease, asthma, multiple sclerosis, periodontitis, space travel, or a combination thereof.

21. (canceled)

22. The method of claim 1, wherein the subject is a human.

23. The method of claim 1, wherein the agent is administered to the subject at one or more doses of from about 0.1 to about 100 mg/kg of body weight of the subject.

24. The method of claim 1, wherein the one or more doses of the agent are administered at least every 1 day, 3 days, 5 days, 1 week, 2 weeks, 3 weeks, or 4 weeks.

25. The method of claim 1, wherein the agent is administered to the subject intratumorally, intravenously, subcutaneously, intraosseously, orally, transdermally, sublingually, in sustained release, in controlled release, in delayed release, or as a suppository.

26. (canceled)

27. (canceled)

28. (canceled)

29. (canceled)

30. (canceled)

31. A composition for increasing bone density or reducing bone loss or bone resorption in a subject in need thereof or treating a disease or disorder associated with reduced bone density, the composition comprising an agent capable of reducing efferocytosis by mesenchymal stromal cells.
